(19) United States
(12) Patent Application Publication
(10) Pub. No.: US 2005/0221305 A1
(43) Pub. Date: Oct. 6, 2005

(54) Antigen panels and methods of using the same

(75) Inventors: Brad Nelson, Victoria (CA); Bradley C. Stone, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Benaroya Research Institute at Virginia Mason, Seattle, WA (US)

(21) Appl. No.: 10/494,874

(22) PCT Filed: Nov. 12, 2002
(86) PCT No.: PCT/US02/36415

§ 371 Date: May 7, 2004

Related U.S. Application Data
(60) Provisional application No. 60/337,983, filed on Nov. 9, 2001.
(60) Provisional application No. 60/368,247, filed on Mar. 27, 2002.

(51) Int. Cl. C12Q 1/68; G01N 33/574

(52) U.S. Cl. 435/6; 435/7.23

(57) ABSTRACT

The present invention provides methods and compositions for the diagnosis, prognosis and treatment of hyperproliferative disease and autoimmune disease. Tumor associated antigens, nucleic acids encoding them and antibodies to the tumor associated antigens are provided for the diagnosis, prognosis and treatment of hyperproliferative disease, such as, for example, ovarian cancer, breast cancer, lung cancer, colorectal cancer, and other epithelial cancers, and for the diagnosis, prognosis and treatment of autoimmune disease.

### CONTINUITY

This application claims the benefit of U.S. Provisional Patent Application No. 60/337,983, filed Nov. 9, 2001, and No. 60/368,247, filed Mar. 27, 2002, the disclosures of which are incorporated by reference herein.

### STATEMENT AS TO RIGHTS TO INVENTIONS MADE UNDER FEDERALLY SPONSORED RESEARCH AND DEVELOPMENT

This work was supported by U.S. Government grants numbers CA82724 and CA84359, awarded by the National Institutes of Health, and U.S. Department of Defense Grant Number OC970002. The U.S. Government may have certain rights in the invention.

### BACKGROUND OF THE INVENTION

Ovarian cancer remains one of the most lethal gynecologic malignancies. It has been reported to be the fifth most common cancer and the fourth leading cause of cancer mortality among women in the United States (see Maller et al. (eds.), SEER Cancer Statistics Review: 1973-1990, National Cancer Institute, Bethesda, Md. (1993)). Epithelial ovarian cancer is the single most lethal gynecological malignancy in the US, with nearly 14,000 deaths annually (see Ries et al. (eds.), SEER Cancer Statistics Review, 1973-1999, National Cancer Institute, Bethesda, Md. (2002)). Over 60% of all women that develop an epithelial-derived ovarian tumor will not survive more than five years beyond initial diagnosis.

The stage of disease at the time of diagnosis is the single most important prognostic factor. If a tumor is localized to the ovary or fallopian tube (stage I/II), the likelihood of achieving a complete cure is over 85% using standard surgery and chemotherapy. In contrast, if the tumor is disseminated (stages III or IV), current treatments yield an expected 5-year survival rate of less than 30%.

Due to the lack of powerful diagnostic tests and also to the absence of any overt symptoms, early detection of ovarian cancer is difficult. Unfortunately, 85% of ovarian cancer patients present with stage III or IV disease at the time of diagnosis. Currently, diagnostic assays are limited to a few markers. Currently, CA125 is the most widely used serum marker for the detection of human ovarian cancer. (Bast et al., J. Clin. Invest. 68:1331-37 (1981); Bast et al., N. Engl. J. Med. 309:883-87 (1983).) However, over 50% of women with early-stage tumors do not display elevated serum CA125, and the false-positive rate of CA125 is high.

Numerous studies on ovarian carcinomas have reported genetic alterations in oncogenes and tumor suppressor genes (see, e.g., Piver et al., Semin. Oncol. 18:177-85 (1991)). Specifically, amplification or activation of the oncogenes HER-2/neu, K-ras and c-myc, as well as inactivation of the tumor suppressor genes p53, BRCA1 and the human mismatch repair genes hMLH1, hMSH2, hPMS1 and hPMS2, have been detected in ovarian cancers. It has been reported that mutation of the p53 gene occurs in about 30-50% of ovarian cancers (see, e.g., Berchuck et al., Am. J. Obstet. Gynecol. 170:246-52 (1994)). p53 gene mutations are common in a variety of other tumors, however.

Recent work in the field of tumor immunology has shown that many cancer patients mount serum antibody responses to tumor-associated antigens at an early stage of disease. One study showed that 30% of patients with Ductal Carcinoma In Situ (DCIS), in which the proto-oncogene HER2/neu was overexpressed, have serum autoantibodies specific for this protein. (Disis et al., J. Clin. Oncol. 15:3363-67 (1997); Disis et al., Cancer Res. 54:16-20 (1994).) In addition, antibodies to p53 have been reported in patients with early stage ovarian, colorectal and oral cancer. (Gadducci et al., Gynecol. Oncol. 72:76-81 (1999); Tang et al., Int. J. Cancer 94:859-63 (2001); Warnakulasuriya et al., J. Pathol. 192:52-57 (2000); Tavassoli et al., Int. J. Cancer 78:390-91 (1998).) Several groups have documented the expression of autoantibodies to NY-ESO-1, p53, HER2/neu, Homeobox-B7 and MAGE family gene products in late stage ovarian cancer patients. (Stockert et al., J. Exp. Med. 187:1349-54 (1998); Vogl et al., Br. J. Cancer 83:1338-43 (2000); Disis et al., Breast Cancer Res. Treat. 62:245-52 (2000); Naora et al., Proc. Natl. Acad. Sci. USA 98:4060-65 (2001).) Whether these responses exist in a significant proportion of early stage ovarian cancer patients remains unknown.

Accordingly, there exists a need to identify new markers associated with ovarian cancer and other epithelial cancers. The present invention satisfies this and other needs.

### BRIEF SUMMARY OF THE INVENTION

The present invention provides methods and compositions for the diagnosis, prognosis and treatment of hyperproliferative disease and autoimmune disease. Tumor associated antigens, nucleic acids encoding them and antibodies to the tumor associated antigens are provided for the diagnosis and prognosis of hyperproliferative disease, such as, for example, ovarian cancer, breast cancer, lung cancer, colorectal cancer, and other epithelial cancers, and for the diagnosis of autoimmune disease.

In one aspect, methods for screening a subject for ovarian cancer are provided. The methods generally include obtaining a sample (e.g., blood or serum) from the subject, the sample comprising antibodies, and determining whether the sample comprises antibodies to at least one tumor associate antigen, such as, for example Topoisomerase II alpha, Werner helicase interacting protein, HEXIM1, HDCMA, Deadbox protein-5, or Kinesin-like 6. The presence of antibodies to at least one tumor associated antigen is correlated with the presence of ovarian cancer in the subject. The method can optionally further include determining whether the sample comprises antibodies to CA125. A sample can also be assayed for the presence of antibodies to tumor associated antigens Topoisomerase II alpha, Ubiquilin-1, Werner helicase interacting protein, p53 and NY-ESO-1, and optionally to tumor associated antigen CA125. In another example, a sample can be assayed for antibodies to Topoisomerase II alpha, Werner helicase interacting protein, HEXIM1, HDCMA, Deadbox protein-5, Ubiquilin-1, HOX-B6, p53 and NY-ESO-1.

In certain embodiments, a sample can be assayed for the presence of antibody to at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of ZFP161, Ubiquilin-1, HOX-B6, IFI27, YB-1, KIAA0136, Osteonectin, F-box only protein 21, and ILF3.

In another aspect, methods for the prognosis or diagnosis of hyperproliferative disease are provided. The methods include obtaining a sample from a subject, the sample including antibodies, and contacting the sample with at least one tumor associated antigen. The tumor associated antigen can be Topoisomerase II alpha, Werner helicase interacting protein, HEXIM1, HDCMA, Deadbox protein-5, and/or Kinesin-like 6. Immunoreaction or complex formation is detected between the tumor associated antigen and the antibodies in the sample. The presence of antibodies to at least one tumor associated antigen is correlated with the presence of ovarian cancer in the subject. A sample can also be assayed for the presence of antibodies to tumor associated antigens Topoisomerase II alpha, Ubiquilin-1, Werner helicase interacting protein, p53 and NY-ESO-1, and optionally to tumor associated antigen CA125. In another example, a sample can be assayed for antibodies to Topoisomerase II alpha, Werner helicase interacting protein, HEXIM1, HDCMA, Deadbox protein-5, Ubiquilin-1, HOX-B6, p53 and NY-ESO-1.

In certain embodiments, tumor associated antigen can be at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of ZFP161, Ubiquilin-1, HOX-B6, IFI27, YB-1, KIAA0136, Osteonectin, F-box only protein 21, and ILF3.

The subject can be, for example, a mammal, such as a human. The sample can be blood, serum, ascites fluid, mucosal fluid, cervical wash, nipple aspirate fluid, stool, urine, saliva, tears, sputum, and the like. Complex formation can be detected by immunoassay, such as, for example, Western blot assay, radioimmunoassay, ELISA, sandwich immunoassay, immunoprecipitation assay, and/or protein A immunoassay. The hyperproliferative disease can be an epithelial cancer, such as for example, ovarian cancer, breast cancer, lung cancer, colorectal cancer, and the like.

In another aspect, methods for prognosis or diagnosis of autoimmune disease in a subject are provided. The methods include obtaining a sample from the subject, the sample including antibodies. The sample is contacted with at least one tumor associated antigen. The tumor associated antigen can be, for example, Topoisomerase II alpha, Werner helicase interacting protein, HEXIM1, HDCMA, Deadbox protein-5, and/or Kinesin-like 6. Complex formation between the tumor associated antigen and the antibodies in the sample is then detected. The presence of antibodies to at least one tumor associated antigen is correlated with the presence of autoimmune disease in the subject. A sample can also be assayed for the presence of antibodies to tumor associated antigens Topoisomerase II alpha, Ubiquilin-1, Werner helicase interacting protein, p53 and NY-ESO-1, and optionally to tumor associated antigen CA125. In another example, a sample can be assayed for antibodies to Topoisomerase II alpha, Werner helicase interacting protein, HEXIM1, HDCMA, Deadbox protein-5, Ubiquilin-1, HOX-B6, p53 and NY-ESO-1.

In certain embodiments, a sample can be assayed for the presence of antibody to at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of ZFP161, Ubiquilin-1, HOX-B6, IFI27, YB-1, KIAA0136, Osteonectin, F-box only protein 21, and ILF3.

The subject can be a mammal, such as a human. The sample can be, for example, blood, serum, ascites fluid, mucosal fluid, cervical wash, nipple aspirate fluid, stool, urine or saliva. Complex formation can be detected by immunoassay, such as, for example, Western blot assay, radioimmunoassay, ELISA, sandwich immunoassay, immunoprecipitation assay, or protein A immunoassay.

The autoimmune disease can be, for example, rheumatoid arthritis, graft versus host disease, systemic lupus erythromatosis (SLE), scleroderma, multiple sclerosis, diabetes, organ rejection, inflammatory bowel disease, psoriasis, and the like.

In yet another aspect, methods for prognosis or diagnosis of hyperproliferative disease in a subject are provided. The methods include obtaining a sample from the subject and contacting the sample with at least one antibody to a tumor associated antigen. The tumor associated antigen can be, for example, Topoisomerase II alpha, Werner helicase interacting protein, HEXIM1, FLJ20267, Deadbox protein-5, and/or Kinesin-like 6. Complex formation between the antibody and tumor associated antigen in the sample is then detected. The presence of antibodies to at least one tumor associated antigen is correlated with the presence of ovarian cancer in the subject. A sample can also be assayed for the presence of antibodies to tumor associated antigens Topoisomerase II alpha, Ubiquilin-1, Werner helicase interacting protein, p53 and NY-ESO-1, and optionally to tumor associated antigen CA125. In another example, a sample can be assayed for antibodies to Topoisomerase II alpha, Werner helicase interacting protein, HEXIM1, HDCMA, Deadbox protein-5, Ubiquilin-1, HOX-B6, p53 and NY-ESO-1.

In certain embodiments, a sample can be assayed for the presence of antibody to at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of ZFP161, Ubiquilin-1, HOX-B6, IFI27, YB-1, KIAA0136, Osteonectin, F-box only protein 21, and ILF3.

The subject can be, for example, a mammal, such as a human. The sample can be tissue, cells, plasma, serum, ascites fluid, mucosal fluid, cervical wash, nipple aspirate fluid, spinal fluid, lymph fluid, the external sections of the skin, respiratory, intestinal, and genitourinary tracts, tears, saliva, hair, tumors, organs, stool, urine, tears, sputum, and the like. Complex formation between the antibody and the antigen can be detected by immunoassay, such as, for example, Western blot assay, radioimmunoassay, ELISA, sandwich immunoassay, immunoprecipitation assay, and/or protein A immunoassay. The hyperproliferative disease can be epithelial cancer, such as, for example, ovarian cancer, breast cancer, lung cancer, colorectal cancer, and the like.

In a related aspect, additional methods for prognosis or diagnosis of hyperproliferative disease in a subject are provided. The methods generally include obtaining a sample from a subject, the sample comprising nucleic acids, and contacting an array of probe molecules stably associated with a surface of a solid support with the sample under hybridization conditions sufficient to produce a hybridization pattern. The probe molecules can include nucleic acids encoding at least a fragment of at least one of a tumor associated antigen, such as, for example, Topoisomerase II alpha, Werner helicase interacting protein, HEXIM1, FLJ20267, Deadbox protein-5, and/or Kinesin-like 6. The hybridization pattern is detected to determine whether the subject has a hyperproliferative disease, wherein the expression pattern is correlated with the presence of hyperproliferative disease in the subject. The sample can be from, for example, ovary, lung, breast or the colorectal tract of the subject. The sample can also be tissue, cells, plasma, serum, ascites fluid, mucosal fluid, cervical wash, nipple aspirate fluid, spinal fluid, lymph fluid, the external sections of the skin, respiratory, intestinal, and genitourinary tracts, tears, sputum, saliva, hair, tumors, organs, stool, or urine. The hyperproliferative disease can be epithelial cancer, such as, for example, ovarian cancer, lung cancer, breast cancer or colorectal cancer. The target nucleic acids can be labeled in one embodiment.

In another embodiment, the tumor associated antigen can be, for example, Topoisomerase II alpha, Ubiquilin-1, Werner helicase interacting protein, p53 and NY-ESO-1, and optionally to tumor associated antigen CA125. In yet another embodiment, the tumor associated antigen can be Topoisomerase II alpha, Werner helicase interacting protein, HEXIM1, HDCMA, Deadbox protein-5, Ubiquilin-1, HOX-B6, p53 and NY-ESO-1.

In yet other embodiments, the tumor associated antigen can be assayed at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of ZFP161, Ubiquilin-1, HOX-B6, IFI27, YB-1, KIAA0136, Osteonectin, F-box only protein 21, and ILF3.

In another aspect, methods are provided for the prognosis or diagnosis of hyperproliferative disease in a subject are provided by determining the methylation profile of a tumor associated antigen gene. The tumor associated antigen gene can be, for example, at least one of Topoisomerase II alpha, Werner helicase interacting protein, HEXIM1, HDCMA, Deadbox protein-5, and/or Kinesin-like 6. The methods generally include obtaining a nucleic acid-containing sample from the subject and determining a methylation profile for a tumor associated antigen gene in the sample. The methylation profile can be compared with a known methylation profile for the tumor associated antigen gene to determine, a prognosis or diagnosis of a risk of hyperproliferative disease in the subject can be made.

In an exemplary embodiment, the methylation profile can be determined by contacting the nucleic acid-containing sample with an agent that modifies unmethylated cytosine, and amplifying the nucleic acid in the sample. The amplified nucleic acids can be examined to determine the methylation profile of the tumor associated gene. The nucleic acid can be amplified with primers that hybridize with a tumor associated antigen gene sequence (e.g., random primers or primers based on a portion of the gene sequence). In certain embodiments, the primers can distinguish between methylated and non-methylated nucleic acid. The methylated nucleic acid in the sample can be detected based on the presence or absence of amplification products produced in the amplification step. The amplifying step can be by, for example, polymerase chain reaction. The modifying agent can be, for example, bisulfite.

In other embodiments, the methylation profile can determined by, for example, digestion with methylation-sensitive restriction enzymes and/or methylation-dependent restriction enzymes.

In related embodiments, the tumor associated antigen gene can encode, for example, Topoisomerase II alpha, Ubiquilin-1, Werner helicase interacting protein, p53 and NY-ESO-1, and optionally tumor associated antigen CA125. In another example, the tumor associated antigen gene can be, for example, Topoisomerase II alpha, Werner helicase interacting protein, HEXIM1, HDCMA, Deadbox protein-5, Ubiquilin-1, HOX-B6, p53 and NY-ESO-1.

In certain embodiments, the tumor associated antigen gene can be encode of human Topoisomerase II alpha, Werner helicase interacting protein, HEXIM1, HDCMA, Deadbox protein-5, and/or Kinesin-like 6, alone or in combination, optionally including another gene encoding at least one of p53, NY-ESO-1, Ubiquilin-1, and/or CA125, and further optionally including another gene encoding at least one of ZFP161, Ubiquilin-1, HOX-B6, IFI27, YB-1, KIAA0136, Osteonectin, F-box only protein 21, and ILF3.

Kits for detecting antibodies to a tumor associated antigen are also provided. A kit can include, for example, at least one tumor associated antigen. The tumor associated antigen can be, for example, Topoisomerase II alpha, Werner helicase interacting protein, HEXIM1, HDCMA, Deadbox protein-5, and/or Kinesin-like 6. In related embodiments, the tumor associated antigen can be, for example, Topoisomerase II alpha, Ubiquilin-1, Werner helicase interacting protein, p53 and NY-ESO-1, and optionally tumor associated antigen CA125. In another example, the tumor associated antigen can be, for example, Topoisomerase II alpha, Werner helicase interacting protein, HEXIM1, HDCMA, Deadbox protein-5, Ubiquilin-1, HOX-B6, p53 and NY-ESO-1. A kit for detecting antibodies to a tumor associated antigen can also include Topoisomerase II alpha, Werner helicase interacting protein, p53 NY-ESO-1, and optionally, Ubiquilin-1 and/or CA125.

In certain embodiments, a kit can include at least one of human Topoisomerase II alpha, Werner helicase interacting protein, HEXIM1, HDCMA, Deadbox protein-5, and/or Kinesin-like 6, alone or in combination, optionally including one or more of p53, NY-ESO-1, Ubiquilin-1, and/or CA125, and further optionally including one or more of p53, NY-ESO-1 and CA125, and at least one of ZFP161, Ubiquilin-1, HOX-B6, IFI27, YB-1, KIAA0136, Osteonectin, F-box only protein 21, and ILF3.

The kits typically further include anti-human antibody for detection of the antigen-antibody complex. In one embodiment, the tumor associated antigen can be labeled; in another embodiment, the anti-human antibody can be labeled.

A kit for detecting expression of tumor associated antigen genes is also provided. The kit can include nucleic acid primers to a tumor associated antigen nucleic acids. The tumor associated antigen nucleic acids can be, for example, Topoisomerase II alpha, Werner helicase interacting protein, HEXIM1, FLJ20267, Deadbox protein-5, and/or Kinesin-like 6. In a related embodiment, the tumor associated antigen nucleic acids can be, for example, Topoisomerase II alpha, Ubiquilin-1, Werner helicase interacting protein, p53 and NY-ESO-1, and optionally tumor associated antigen CA125. In another embodiment, the tumor associated antigen nucleic acids can be, for example, Topoisomerase II alpha, Werner helicase interacting protein, HEXIM1, HDCMA, Deadbox protein-5, Ubiquilin-1, HOX-B6, p53 and NY-ESO-1. In yet another embodiment, the tumor associated antigen nucleic acid can encode, for example, Topoisomerase II alpha, Werner helicase interacting protein, p53 NY-ESO-1, and optionally, Ubiquilin-1 and/or CA125.

In yet additional embodiments, a kit can include nucleic acids encoding at least one of human Topoisomerase II alpha, Werner helicase interacting protein, HEXIM1, HDCMA, Deadbox protein-5, and/or Kinesin-like 6, alone or in combination, optionally encoding one or more of p53, NY-ESO-1, Ubiquilin-1, and/or CA125, and further optionally encoding at least one of ZFP161, Ubiquilin-1, HOX-B6, IFI27, YB-1, KIAA0136, Osteonectin, F-box only protein 21, and ILF3.

The kit typically further includes a polynucleotide polymerase, nucleotides, and/or a buffer. The kit can optionally further include an array of probe molecules for use in a hybridization assay.

In another aspect, methods for treating a subject having an hyperproliferative disease are provided. The methods generally include administering the subject an effective amount of an immunotherapeutic composition effective to reduce or ameliorate the hyperproliferative disease. The immunotherapeutic composition can be, for example, at least one of Topoisomerase II alpha, Werner helicase interacting protein, HEXIM1, HDCMA, Deadbox protein-5 and Kinesin-like 6; antibodies to at least one of Topoisomerase II alpha, Werner helicase interacting protein, HEXIM1, HDCMA, Deadbox protein-5 and Kinesin-like 6; or T cells to at least one of Topoisomerase II alpha, Werner helicase interacting protein, HEXIM1, HDCMA, Deadbox protein-5 and Kinesin-like 6, and the like.

### BRIEF DESCRIPTION OF THE DRAWINGS

FIG. 1 shows a summary of SEREX array screening, ELISA and CA125 radioimmunoassay results for sera from cancer patient serum panels #1 and #2 and normal controls. Antigens and their chromosomal locations are listed in the two leftmost columns, assay results for individual patients from serum panel #2 are shown in the 25 central columns, and numeric summaries of these data are provided in the three columns listed under Patient Serum Summary. A summary of responses found in normal controls is provided in the far right column. Black boxes indicate a positive score for autoantibodies to a given antigen by SEREX array and ELISA. Diagonally striped boxes indicate a positive autoantibody score determined by ELISA (where positive is defined as an optical density greater than the mean plus 3 standard deviations for cancer-free controls). Checkered boxes indicate patients with a positive CA125 result determined by radioimmunoassay using the standard clinical threshold of >35 units/ml. Clear boxes indicate a negative score. Twenty normal controls consisting of women without a personal history of cancer or autoimmune disease were negative by for autoantibodies to these antigens by SEREX array analysis, while forty-five normal controls scored negative by ELISA. ND means not determined, NA means not applicable.

FIG. 2 shows real-time RT-PCR analysis of mRNA expression levels for tumor associated antigens encoded on chromosome 17q, as well as MUC16/CA125 and the housekeeping gene TMP21. Analysis was carried out with 22 normal tissue samples representing 19 different tissues, 8 samples of normal whole ovary, 8 samples of benign ovarian tumors, 23 late stage and 3 early stage ovarian tumors. Relative mRNA abundance is shown on a log scale using arbitrary units, which cannot be directly compared between different genes. Values of zero were not graphed. Individual early stage tumors are numbered 1-3.

FIG. 3 shows real-time RT-PCR analysis of mRNA expression levels for tumor associated antigens UBQLN1, RUVBL, HDCMA, p53 and NY-ESO-1. The study design and data presentation are the same as for FIG. 2.

FIG. 4 shows the results of ELISA's of HIS1 and GAPDH proteins levels in samples of benign breast lesions, early stage breast cancer, later stage breast cancer and normal controls.

### DEFINITIONS

Prior to setting forth the invention in more detail, it may be helpful to a further understanding thereof to set forth definitions of certain terms as used hereinafter. Unless defined otherwise, all technical and scientific terms used herein have the same meaning as commonly understood by one of ordinary skill in the art to which this invention belongs. Only exemplary methods and materials are described, and any methods and materials similar to those described herein can be used in the practice or testing of the present invention. For purposes of the present invention, the following terms are defined below.

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA, synthetic forms, and mixed polymers, both sense and antisense strands, and can also be chemically or biochemically modified or can contain non-natural or derivatized nucleotide bases, as will be readily appreciated by the skilled artisan. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “oligonucleotide” refers to a polynucleotide of from about six (6) to about one hundred (100) nucleotides or more in length. Thus, oligonucleotides are a subset of polynucleotides. Oligonucleotides can be synthesized, for example, on an automated oligonucleotide synthesizer (for example, those manufactured by Applied BioSystems (Foster City, Calif.)), according to specifications provided by the manufacturer.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “polypeptide” refers to a polymer of amino acids and its equivalent and does not refer to a specific length of the product; thus, peptides, oligopeptides and proteins are included within the definition of a polypeptide. A fragment refers to a portion of a polypeptide having at least 6 contiguous amino acids, typically 8-10 contiguous amino acids, more typically at least 20 contiguous amino acids, still more typically at least 50 contiguous amino acids of, for example, a tumor associated antigen polypeptide. A derivative is a polypeptide having conservative amino acid substitutions, as compared with another sequence. Derivatives further include, for example, glycosylations, acetylations, phosphorylations, and the like. Further included are analogs of polypeptides containing one or more analogs of an amino acid (e.g., unnatural amino acids, and the like), polypeptides with substituted linkages as well as other modifications known in the art, both naturally and non-naturally occurring, as more fully described infra.

The terms “amino acid” or “amino acid residue”, as used herein, refer to naturally occurring L amino acids or to D amino acids. The commonly used one- and three-letter abbreviations for amino acids are used herein (see, e.g., Alberts et al., Molecular Biology of the Cell, Garland Publishing, Inc., New York (3d ed. 1994)).

The term “heterologous” refers to a nucleic acid or polypeptide from a different source, such as a different protein, tissue, organism or species, as compared with another nucleic acid or polypeptide.

The term “isolated” refers to a nucleic acid, polypeptide or antibody that has been removed from its natural cellular environment. An isolated nucleic acid is typically at least partially purified from other cellular nucleic acids, polypeptides and other constituents.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised to the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 60%, 70%, 75%, 80%, 85%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences indicates that the polypeptide comprises a sequence with at least 70% sequence identity to a reference sequence, or typically 80%, or more typically 85% sequence identity or 90% sequence identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

The term “conservative substitution,” when describing a polypeptide, refers to a change in the amino acid composition of the polypeptide that does not substantially alter the polypeptide's activity and/or antigenicity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not substantially alter activity and/or antigenicity. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482 (1981)), by the homology alignment algorithm of Needleman and Wunsch (J. Mol. Biol. 48:443 (1970)), by the search for identity method of Pearson and Lipman (Proc. Natl. Acad. Sci. USA 85:2444 (1988)), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology, 4th ed., John Wiley and Sons, New York (1999)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (J. Mol. Evol. 35:351-60 (1987)). The method used is similar to the CLUSTAL method described by Higgins and Sharp (Gene 73:237-44 (1988); CABIOS 5:151-53 (1989)). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described in Altschul et al. (J. Mol. Biol. 215:403-10 (1990)). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) can use as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin and Altschul, Proc. Natl. Acad. Sci. USA 90:5873-87 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is typically less than about 0.1, more typically less than about 0.01, and most typically less than about 0.001. Another indication that two nucleic acids are substantially identical is that the two molecules hybridize specifically to each other under stringent conditions.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization conditions” and “stringent hybridization wash conditions” in the context of nucleic acid hybridization studies, such as Southern and northern hybridization, are sequence-dependent, and are different under different environmental parameters. Longer sequences hybridize specifically at higher temperatures. A guide to the hybridization of nucleic acids is found in Tijssen, Laboratory Techniques in Biochemistry and Molecular Biology—Hybridization with Nucleic Acid Probes (part I, chapter 2 “Overview of principles of hybridization and the strategy of nucleic acid probe assays,” Elsevier, N.Y. (1993), which is incorporated by reference herein). Generally, highly stringent hybridization and wash conditions are selected to be about 5° C. lower than the thermal melting point (Tₘ) for the specific sequence at a defined ionic strength and pH. Typically, under “stringent conditions,” a probe will hybridize to its target subsequence, but not to other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide in 4-6×SSC or SSPE at 42° C., or 65-68° C. in aqueous solution containing 4-6×SSC or SSPE. An example of highly stringent wash conditions is 0.15 M NaCl at 72° C. for about 15 minutes. An example of stringent wash conditions is a 0.2×SSC wash at 65° C. for 15 minutes. (See generally Sambrook et al., Molecular Cloning, A Laboratory Manual, 3rd ed., Cold Spring Harbor Publish., Cold Spring Harbor, N.Y. (2001), which is incorporated by reference herein.) Often, a high stringency wash is preceded by a low stringency wash to remove background probe signal. An example of medium stringency wash for a duplex of, for example, more than 100 nucleotides, is 1×SSC at 45° C. for 15 minutes. An example of a low stringency wash for a duplex of, for example, more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code.

The term “immunologically cross-reactive” means that a polypeptide, fragment, derivative or analog is capable of competitively inhibiting the binding of an antibody to its antigen.

The term “sample” generally indicates a specimen of tissue, cells, plasma, serum, ascites fluid, mucosal fluid, cervical wash, nipple aspirate fluid, spinal fluid, lymph fluid, the external sections of the skin, respiratory, intestinal, and genitourinary tracts, tears, saliva, hair, tumors, organs, stool, urine, other material of biological origin that contains antibodies, polypeptide and/or polynucleotides, or in vitro cell culture constituents of any of these. A sample can further be semi-purified or purified forms of antibodies, polypeptides and/or polynucleotides. A sample can be isolated from a mammal, such as a human, an animal, any other organism as well as in vitro culture constituents of any of these.

The term “proliferation” refers to activities such as growth, reproduction, change in gene expression, transformation, and other changes in cell state. “Hyperproliferation” refers to an increase in one or more proliferative activities, as compared with normal cells or tissue. “Hyperproliferative disease” refers to a disease, condition, or disorder associated with hyperproliferation of cells or tissues in a subject. Diseases involving hyper-proliferation include, but are not limited to, cancer, malignancies, premalignant conditions (e.g., hyperplasia, metaplasia, dysplasia), benign tumors, hyperproliferative disorders, benign dysproliferative disorders, autoimmune diseases, and the like.

The term “antibody” refers to a polypeptide substantially encoded by an immunoglobulin gene or immunoglobulin genes, or fragments thereof, that specifically binds and recognizes an analyte (e.g., antigen). Immunoglobulin genes include the kappa, lambda, alpha, gamma, delta, epsilon and mu constant region genes, as well as the myriad immunoglobulin variable region genes. Immunoglobulins can be, for example, IgG, IgM, IgA, IgD, IgE, and the like.

Antibodies exist, for example, as intact immunoglobulins or as a number of well characterized antigen-binding fragments, such as, for example, F(ab′)₂ fragments, Fab′ fragments, Fab fragments, an antigen binding Fv fragment, a single heavy chain or a chimeric antibody. Such antibodies can be produced by the modification of whole antibodies or synthesized de novo using recombinant DNA methodologies. (See, e.g., Harlow and Lane, Using Antibodies, A Laboratory Manual, Cold Spring Harbor Laboratory, New York (1999), the disclosure of which is incorporated by reference herein.)

### DESCRIPTION OF THE SPECIFIC EMBODIMENTS

The present invention provides methods and compositions for the diagnosis, prognosis and treatment of hyperproliferative disease and/or autoimmune disease. Tumor associated antigens, nucleic acids encoding them, and antibodies to the tumor associated antigens are provided for the prognosis, diagnosis and/or treatment of hyperproliferative disease, such as, for example, ovarian cancer, breast cancer, lung cancer, colorectal cancer, and other epithelial cancers, and/or autoimmune disease.

Tumor Associated Antigen Nucleic Acids

In one aspect, nucleic acids encoding tumor associated antigens are provided as markers of hyperproliferative disease or autoimmune disease. Such tumor associated antigen nucleic acids can encode, for example, proteins or fragments, derivatives and analogs thereof, the function (e.g., expression or activity) of which is altered in cells associated with hyperproliferative disease and/or autoimmune disease. The tumor associated antigen nucleic acids can also encode polypeptides of normal function, but which are differentially immunogenic in the context of cells associated with hyperproliferative disease and/or autoimmune disease, as compared with normal epithelial cells of the same tissue or cell type.

The tumor associated antigen nucleic acids can include, for example, nucleic acids encoding Topoisomerase II alpha (also referred to as TOP2a), Werner helicase interacting protein (also referred to as RUVBL or and WHIP), HEXIM1 (also referred to as HMBA-inducible protein 1 or HIS1), FLJ20267 (also referred to as HDCMA18P or HDCMA), Deadbox protein-5 (also referred to as DDX5 or Dead/H (Asp-Glu-Ala-Asp/His) polypeptide-5), and/or Kinesin-like 6 (also referred to as KNSL6 or mitotic centromere associated Kinesin). The tumor associated antigen nucleic acids can further include those encoding the tumor suppressor gene p53, the cancer-testis antigen NY-ESO-1, Ubiquilin-1, and/or CA125.

The tumor associated antigen nucleic acids can also be at least one of p53, NY-ESO-1, Ubiquilin-1, and/or HOX-B6. In a related embodiment, the tumor associated antigen nucleic acids can be at least one of Topoisomerase II alpha, Ubiquilin-1, Werner helicase interacting protein, p53 and NY-ESO-1, and optionally tumor associated antigen CA125. In another embodiment, the tumor associated antigen nucleic acids can be at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, the tumor associated antigen nucleic acids can be at least one of Topoisomerase II alpha, Werner helicase interacting protein, p53, NY-ESO-1, and optionally, Ubiquilin-1 and/or CA125.

In a further embodiment, tumor associated antigen nucleic acids can be at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3. (See, e.g., U.S. patent application Ser. No. 10/106,559, filed Mar. 25, 2002; the disclosure of which is incorporated by reference herein).

In a related embodiment, the tumor associated antigen nucleic acids can be, for example, Topoisomerase II alpha, Ubiquilin-1, Werner helicase interacting protein, p53 and NY-ESO-1, and optionally tumor associated antigen CA125. In another embodiment, the tumor associated antigen nucleic acids can be, for example, Topoisomerase II alpha, Werner helicase interacting protein, HEXIM1, HDCMA, Deadbox protein-5, Ubiquilin-1, HOX-B6, p53 and NY-ESO-1. In yet another embodiment, the tumor associated antigen nucleic acid can be, for example, Topoisomerase II alpha, Werner helicase interacting protein, p53 NY-ESO-1, and optionally, Ubiquilin-1 and/or CA125.

Such tumor associated antigen nucleic acids can include nucleic acids from human and non-human mammals, such as, for example, porcine, bovine, feline, equine, and/or canine species, as well as primate species.

In certain embodiments, the tumor associated antigen nucleic acids correspond to human nucleic acid sequences encoding Topoisomerase II alpha, Werner helicase interacting protein, HEXIM1, FLJ20267, Deadbox protein-5, and/or Kinesin-like 6. The tumor associated antigen nucleic acids can further include those encoding the tumor suppressor gene p53, the cancer-testis antigen NY-ESO-1, Ubiquilin-1, and/or HOX-B6. In specific embodiments, the tumor associated antigen nucleic acids correspond to the following nucleic acids, which are referenced by their National Center for Biotechnology Information Unigene accession numbers: Topoisomerase II alpha (Hs.156346; TOP2a); Werner helicase interacting protein (Hs.236828; WHIP); HEXIM1 (Hs.15299; HIS1); FLJ20267 (Hs.278635; HDCMA18P); Deadbox protein-5 (Hs.76053; DDX5); Kinesin-like 6 (Hs.69630; KNSL6), or the coding regions thereof. The tumor associated antigen nucleic acids can additionally correspond to the nucleic acids encoding p53 (Hs.1846; TP53); NY-ESO-1 (Hs. 167379; CTAG1); Ubiquilin-1 (Hs.9589; UBQLN1); and/or HOX-B6 (Hs.98428; HOXB6), or the coding regions thereof (All of these sequences are incorporated by reference herein in their entirety.) In additional specific embodiments, the tumor associated antigen nucleic acids correspond to the following nucleic acids, which are referenced by their National Center for Biotechnology Information Unigene accession numbers: ZFP161 (Hs.156000; ZFP161); Ubiquilin-1 (Hs.9589; UBQLN1); HOX-B6 (Hs.98428; HOXB6); IFI27 (Hs.278613; IFI27); YB-1 (Hs.74497; NSEP1); KIAA0136 (Hs.70359; KIAA0316); Osteonectin (Hs.111779; SPARC); F-box only protein 21 (Hs.184227; FBXO21); ILF3 (Hs.256583; ILF3), or the coding regions thereof.

The invention also provides fragments of tumor associated antigen nucleic acids comprising at least 6 contiguous nucleotides (i.e., a hybridizable portion); in other embodiments, the nucleic acids comprise contiguous nucleotides of at least 10 nucleotides, 25 nucleotides, 50 nucleotides, 100 nucleotides, 150 nucleotides, 200 nucleotides, or 250 nucleotides of a tumor associated antigen nucleic acid sequence. The nucleic acids can also be smaller than 35, 200 or 250 nucleotides in length. These nucleic acids can be single or double stranded. As used herein, a “nucleic acid encoding a fragment or portion of a tumor associated antigen polypeptide” refers to a nucleic acid encoding only the recited fragment or portion of the tumor associated antigen polypeptide and not the other contiguous portions of the tumor associated antigen polypeptide as a continuous sequence. Fragments of tumor associated antigen nucleic acids comprising regions conserved between other tumor associated antigen nucleic acids, of the same or different species, are also provided. Nucleic acids encoding one or more tumor associated antigen domains are also provided.

The invention also relates to nucleic acids hybridizable to or complementary to the foregoing sequences. In specific aspects, nucleic acids are provided which comprise a sequence complementary to at least 10, 25, 50, 100, 200, or 250 nucleotides of a tumor associated antigen gene, or a portion thereof. In a specific embodiment, a nucleic acid which is hybridizable to a tumor associated antigen nucleic acid, or to a nucleic acid encoding a tumor associated antigen derivative, under conditions of low, medium or high stringency is provided. Low, moderate and high stringency conditions are well known to those of skill in the art, and will vary predictably depending on the base composition of the particular nucleic acid sequence and on the specific organism from which the nucleic acid sequence is derived. For guidance regarding such conditions see, for example, Sambrook et al. (supra); Ausubel et al. (supra) and Tijssen (supra) (all of which are incorporated by reference herein).

Nucleic acids encoding derivatives and analogs of tumor associated antigen proteins, and tumor associated antigen antisense nucleic acids are additionally provided. Derivatives of the tumor associated antigen sequences include those nucleotide sequences encoding substantially the same amino acid sequences as found in native tumor associated antigen proteins, and those encoded amino acid sequences with functionally equivalent amino acids (e.g., conservative substitutions).

Tumor associated antigen nucleic acids can be obtained by standard procedures known in the art (e.g., by chemical synthesis, by cDNA cloning, by the cloning of genomic DNA, by PCR amplification, and the like). (See, e.g., Sambrook et al., supra; Glover (ed.), DNA Cloning: A Practical Approach, MRL Press, Ltd., Oxford, U.K. Vol. I, II (1995); Ausubel et al., supra; the disclosures of which are incorporated by reference herein.) The nucleic acids can also identified by searching nucleic databases for nucleic acid sequences that are substantially similar to known tumor associated antigen nucleic acid sequences.

Tumor Associated Antigen Polypeptides

In another aspect, the invention relates to tumor associated antigen polypeptides markers of hyperproliferative disease, such as epithelial cancers (e.g., epithelial ovarian cancer). The invention further relates to tumor associated antigen polypeptides markers of autoimmune disease. Such tumor associated antigen polypeptides can include, for example, proteins, or fragments, derivatives and analogs thereof, the function (e.g., expression or activity) of which is altered in cells associated with hyperproliferative disease and/or autoimmune disease. The tumor associated antigens also include polypeptides of normal function, but which are differentially immunogenic in cells associated with hyperproliferative disease and/or autoimmune disease as compared with normal epithelial cells of the same tissue or cell type.

Tumor associated antigen polypeptides include, for example, one or more of Topoisomerase II alpha, Werner helicase interacting protein, HEXIM1, HDCMA, Deadbox protein-5, and/or Kinesin-like 6, alone or in combination. Tumor associated antigen polypeptides can further optionally include at least one of p53, NY-ESO-1, Ubiquilin-1, and/or HOX-B6, and/or fragments, derivatives or analogs of any of these, as further discussed below. Tumor associated antigens can include polypeptides from human and non-human mammals, such as, for example, porcine, bovine, feline, equine, and/or canine species, as well as other primate species.

In certain embodiments, the tumor associated antigen polypeptides are human Topoisomerase II alpha, Werner helicase interacting protein, HEXIM1, HDCMA, Deadbox protein-5, and/or Kinesin-like 6, alone or in combination, and/or fragments, derivative or analogs thereof. In another embodiment, the tumor associated antigens can optionally further include at least one of p53, NY-ESO-1, Ubiquilin-1, and/or CA125, and/or fragments, derivative or analogs thereof. In yet another embodiment, the tumor associate antigens can include Topoisomerase II alpha, Werner helicase interacting protein, p53 and NY-ESO-1, or Topoisomerase II alpha, Werner helicase interacting protein, p53, NY-ESO-1 and Ubiquilin-1, and optionally CA125. In still another embodiment, the tumor associated antigens include Topoisomerase II alpha, Werner helicase interacting protein, Deadbox protein-5, HEXIM1, HDCMA, Ubiqulin-1, HOX-B6, p53, and NY-ESO-1, and/or fragments, derivative or analogs thereof.

The tumor associated antigen polypeptides acids can also be at least one of p53, NY-ESO-1, Ubiquilin-1, and/or HOX-B6. In a related embodiment, the tumor associated antigen polypeptides can be at least one of Topoisomerase II alpha, Ubiquilin-1, Werner helicase interacting protein, p53 and NY-ESO-1, and optionally tumor associated antigen CA125. In another embodiment, the tumor associated antigen polypeptides can be at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, the tumor associated antigen polypeptides can be at least one of Topoisomerase II alpha, Werner helicase interacting protein, p53, NY-ESO-1, and optionally, Ubiquilin-1 and/or CA125.

In a further embodiment, tumor associated antigen polypeptides can be at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

In specific embodiments, the tumor associated antigen polypeptides have the deduced amino acid sequences of the following tumor associated antigen nucleic acid sequences (which are referenced by their National Center for Biotechnology Information Unigene accession numbers): Topoisomerase II alpha (Hs.156346; TOP2a); Werner helicase interacting protein (Hs.236828; WHIP); HEXIM1 (Hs.15299; HIS1); FLJ20267 (HDCMA) (Hs.278635; HDCMA18P); Deadbox protein-5 (Hs.76053; DDX5); and/or Kinesin-like 6 (Hs.69630; KNSL6). The tumor associated antigens can also have the deduced amino acid sequences of p53 (Hs.1846; TP53), NY-ESO-1 (Hs. 167379; CTAG1); Ubiquilin-1 (Hs.9589; UBQLN1); HOX-B6 (Hs.98428; HOXB6), or fragments thereof.

In additional specific embodiments, the tumor associated antigens have the deduced amino acid sequences of the following tumor associated antigen nucleic acid sequences (which are referenced by their National Center for Biotechnology Information Unigene accession numbers): Ubiquilin-1 (Hs.9589; UBQLN1); IFI27 (Hs.278613; IFI27); HOX-B6 (Hs.98428; HOXB6); ZFP161 (Hs.156000; ZFP161); YB-1 (Hs.74497; NSEP1); KIAA0136 (Hs.70359; KIAA0316); Osteonectin (Hs.111779; SPARC); F-box only protein 21 (Hs.184227; FBXO21); and/or ILF3 (Hs.256583; ILF3). The tumor associated antigens can also have the deduced amino acid sequences of p53 (Hs.1846; TP53) and/or NY-ESO-1 (Hs. 167379; CTAG1), or fragments thereof.

Tumor associated antigen polypeptide derivatives include naturally-occurring amino acid sequence variants as well as those altered by substitution, addition or deletion of one or more amino acid residues. Tumor associated antigen polypeptide derivatives include, but are not limited to, those containing as a primary amino acid sequence of all or part of the amino acid sequence of a tumor associated antigen polypeptide including altered sequences in which one or more functionally equivalent amino acid residues (e.g., a conservative substitution) are substituted for residues within the sequence, resulting in a silent change.

In another aspect, a polypeptide consisting of or comprising an antigenic fragment of a tumor associated antigen polypeptide having at least 10 contiguous amino acids of the tumor associated antigen polypeptide is provided. In other embodiments, the antigenic fragment has at least 20 or 50 contiguous amino acids of the tumor associated antigen polypeptide. The fragments can also be smaller than 35, 100 or 200 amino acids.

Antigenic fragments, derivatives or analogs of tumor associated antigen polypeptides include, but are not limited to, those molecules comprising regions that are substantially similar to tumor associated antigen polypeptide or fragments thereof (e.g., in various embodiments, at least 70%, 75%, 80%, 90%, or even 95% identity or similarity over an amino acid sequence of identical size), or when compared to an aligned sequence in which the alignment is done by a computer sequence comparison/alignment program known in the art, or whose coding nucleic acid is capable of hybridizing to a tumor associated antigen nucleic acid, under high stringency, moderate stringency, or low stringency conditions (supra).

Tumor associated antigen polypeptide fragments, derivatives and analogs can be produced by various methods known in the art. The manipulations which result in their production can occur at the gene or protein level. For example, a cloned tumor associated antigen nucleic acid can be modified by any of numerous strategies known in the art (see, e.g., Sambrook et al., supra), such as making conservative substitutions, deletions, insertions, and the like. The sequence can be cleaved at appropriate sites with restriction endonuclease(s), followed by further enzymatic modification if desired, isolated, and ligated in vitro. (See generally Ausubel et al. (supra) and Sambrook et al. (supra).) In the production of the tumor associated antigen nucleic acids encoding a polypeptide, fragment, derivative or analog of a tumor associated antigen polypeptide, the modified nucleic acid typically remains in the proper translational reading frame, so that the reading frame is not interrupted by translational stop signals or other signals which interfere with the synthesis of the tumor associated antigen fragment, derivative or analog. Tumor associated antigen nucleic acids can also be mutated in vitro or in vivo to create and/or destroy translation, initiation and/or termination sequences. The tumor associated antigen encoding nucleic acid can also be mutated to create variations in coding regions and/or to form new restriction endonuclease sites or destroy preexisting ones and to facilitate further in vitro modification. Any technique for mutagenesis known in the art can be used, including but not limited to, chemical mutagenesis, in vitro site-directed mutagenesis (see, e.g., Hutchison et al., J. Biol. Chem. 253:6551-60 (1978); Sambrook et al., supra), and the like.

Manipulations of the tumor associated antigen polypeptide sequence can also be made at the polypeptide level. Included within the scope of the invention are tumor associated antigen polypeptide fragments, derivatives or analogs which are differentially modified during or after synthesis (e.g., in vivo or in vitro translation). The polypeptide sequences can be modified to increase and/or decrease antigenicity, as will be appreciated by the skilled artisan. Such modifications include conservative substitution, glycosylation, acetylation, phosphorylation, amidation, derivatization by known protecting/blocking groups, proteolytic cleavage, linkage to an antibody molecule or other cellular ligand, and the like. Any of numerous chemical modifications can be carried out by known techniques, including, but not limited to, specific chemical cleavage (e.g., by cyanogen bromide), enzymatic cleavage (e.g., by trypsin, chymotrypsin, papain, V8 protease, and the like); modification by, for example, NaBH₄ acetylation, formylation, oxidation and reduction, metabolic synthesis in the presence of tunicamycin, and the like.

In addition, tumor associated antigen polypeptides, fragments, derivatives and analogs can be chemically synthesized. For example, a peptide corresponding to a portion, or antigenic fragment, of a tumor associated antigen polypeptide, which comprises a desired domain, or which has the desired antigenicity, can be synthesized by chemical synthetic methods using, for example, an automated peptide synthesizer. Furthermore, if desired, nonclassical amino acids or chemical amino acid analogs can be introduced as a substitution or addition into the tumor associated antigen polypeptide sequence. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

In a specific embodiment, the tumor associated antigen polypeptide, fragment, derivative or analog is a chimeric, or fusion, protein comprising a tumor associated antigen polypeptide, fragment, derivative or antigen thereof (typically containing at least a domain or motif of the tumor associated antigen polypeptide, or at least 10 contiguous amino acids of the tumor associated antigen polypeptide) joined at its amino- or carboxy-terminus via a peptide bond to an amino acid sequence of a different protein. In one embodiment, such a chimeric protein is produced by recombinant expression of a nucleic acid encoding the fusion protein. The chimeric product can be made by ligating the appropriate nucleic acid sequences, encoding the desired amino acid sequences, to each other in the proper coding frame and expressing the chimeric product by methods commonly known in the art. Alternatively, the chimeric product can be made by protein synthetic techniques (e.g., by use of an automated peptide synthesizer).

The production and use of tumor associated antigen polypeptides, fragments, derivatives and analogs thereof are also within the scope of the present invention. In a specific embodiment, the polypeptide, fragment, derivative or analog is immunogenic or antigenic (e.g., that can be recognized by an antibody specific for the tumor associated antigen polypeptide or by immune cell such as T cells). As one example, such fragments, derivatives or analogs which have the desired immunogenicity or antigenicity can be used, for example, in immunoassays, for immunization, and the like. A specific embodiment relates to a tumor associated antigen fragment that can be bound by an anti-tumor associated antigen antibody, such as an antibody in a sample from a subject. Fragments, derivatives or analogs of tumor associated antigen can be tested for the desired activity by methods known in the art.

Tumor associated antigen polypeptides can be isolated and purified by standard methods including chromatography (e.g., ion exchange, affinity, sizing column chromatography, high pressure liquid chromatography, and like), centrifugation, differential solubility, or by any other standard technique for the purification of proteins. The functional properties can be evaluated using any suitable assay as described herein or otherwise known to the skilled artisan. Alternatively, a tumor associated antigen polypeptide can be produced by a recombinant host cell. The protein can be synthesized by standard chemical methods known in the art (see, e.g., Hunkapiller et al., Nature 310:105-11 (1984); Stewart and Young, Solid Phase Peptide Synthesis, 2nd Ed., Pierce Chemical Co., Rockford, Ill., (1984)). In another alternate embodiment, native tumor associated antigen polypeptides can be purified from natural sources by standard methods such as those described above (e.g., immunoaffinity purification). In a specific embodiment, tumor associated antigen polypeptides, whether produced by recombinant DNA techniques, by chemical synthetic methods or by purification of native polypeptides, include but are not limited to those containing as a primary amino acid sequence all or part of the amino acid sequence of tumor associated antigen polypeptide, as well as fragments, derivatives and analogs thereof.

Antibodies to Tumor Associated Antigens

Antibodies to tumor associated antigens are also provided. The antibodies are typically to a tumor associated antigen, such as, for example, at least one of Topoisomerase II alpha, Werner helicase interacting protein, HEXIM1, FLJ20267, Deadbox protein-5, and/or Kinesin-like 6, or fragments, derivative or analogs thereof. The antibodies can further include those for at least one of p53, NY-ESO-1, Ubiquilin-1, HOX-B6, or fragments, derivative or analogs thereof. In specific embodiments, the antibodies are to human tumor associated antigens.

In a related embodiment, the antibodies are to Topoisomerase II alpha, Ubiquilin-1, Werner helicase interacting protein, p53 and NY-ESO-1, and optionally tumor associated antigen CA125. In another embodiment, antibodies are to Topoisomerase II alpha, Werner helicase interacting protein, HEXIM1, HDCMA, Deadbox protein-5, Ubiquilin-1, HOX-B6, p53 and NY-ESO-1. In yet another embodiment, the antibodies are to Topoisomerase II alpha, Werner helicase interacting protein, p53 NY-ESO-1, and optionally, Ubiquilin-1 and/or CA125.

In other embodiments, the antibodies are to at least one of p53, NY-ESO-1, Ubiquilin-1, and/or HOX-B6. In a related embodiment, the antibodies are to at least one of Topoisomerase II alpha, Ubiquilin-1, Werner helicase interacting protein, p53 and NY-ESO-1, and optionally tumor associated antigen CA125. In another embodiment, the antibodies are to at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, the antibodies are to at least one of Topoisomerase II alpha, Werner helicase interacting protein, p53, NY-ESO-1, and optionally, Ubiquilin-1 and/or CA125.

In a further embodiment, the antibodies are to at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

Tumor associated antigen antibodies include, but are not limited to, polyclonal antibodies, monoclonal antibodies, chimeric antibodies, single chain antibodies, antigen binding antibody fragments (e.g., Fab, Fab′, F(ab′)₂, Fv, or hypervariable regions), bi-specific antibodies, and an Fab expression library. In certain embodiments, polyclonal and/or monoclonal antibodies to a tumor associated antigen are produced. In other embodiments, antibodies to a domain of a tumor associated antigen are produced. In yet other embodiments, fragments of a tumor associated antigen that are identified as immunogenic are used as immunogens for antibody production.

Various procedures known in the art can be used for the production of polyclonal antibodies. For the production of such antibodies, various host animals (including, but not limited to, rabbits, mice, rats, sheep, goats, camels, and the like) can be immunized by injection with a tumor associated antigen, fragment, derivative or analog. Various adjuvants can be used to increase the immunological response, depending on the host species. Such adjuvants include, for example, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and other adjuvants, such as BCG (bacille Calmette-Guerin) and Corynebacterium parvum.

For preparation of monoclonal antibodies directed toward a tumor associated antigen, suitable techniques include those that provide for the production of antibody molecules. Such techniques include, for example, the hybridoma technique originally developed by Kohler and Milstein (see, e.g., Nature 256:495-97 (1975)), the trioma technique (see, e.g., Hagiwara and Yuasa, Hum. Antibodies Hybridomas 4:15-19 (1993); Hering et al., Biomed. Biochim. Acta 47:211-16 (1988)), the human B-cell hybridoma technique (see, e.g., Kozbor et al., Immunology Today 4:72 (1983)), and the EBV-hybridoma technique to produce human monoclonal antibodies (see, e.g., Cole et al., In: Monoclonal Antibodies and Cancer Therapy, Alan R. Liss, Inc., pp. 77-96 (1985)). Human antibodies also can be used and can be obtained by using human hybridomas (see, e.g., Cote et al., Proc. Natl. Acad. Sci. USA 80:2026-30 (1983)) or by transforming human B cells with EBV virus in vitro (see, e.g., Cole et al., supra). (See generally Harlow and Lane (supra).)

Further to the invention, “chimeric” antibodies (see, e.g., Morrison et al., Proc. Natl. Acad. Sci. USA 81:6851-55 (1984); Neuberger et al., Nature 312:604-08 (1984); Takeda et al., Nature 314:452-54 (1985)) can be prepared. Such chimeric antibodies are typically prepared by splicing the genes (of one species) for an antibody molecule specific for tumor associated antigen together with genes from another species of antibody molecule of appropriate biological activity. It can be desirable to transfer the antigen binding regions (e.g., Fab′, F(ab′)₂, Fab, Fv, or hypervariable regions) of antibodies from one species into the framework of an antibody from another species by recombinant DNA techniques to produce a chimeric molecule. Methods for producing such “chimeric” molecules are generally well known and described in, for example, U.S. Pat. Nos. 4,816,567; 4,816,397; 5,693,762; and 5,712,120; PCT Patent Publications WO 87/02671 and WO 90/00616; and European Patent Publication EP 239 400 (the disclosures of which are incorporated by reference herein). In a specific embodiment, a human monoclonal antibody or portions thereof can be identified by screening a human B-cell cDNA library for nucleic acid molecules that encode antibodies that specifically bind to a tumor associated antigen according to the method generally set forth by Huse et al. (Science 246:1275-81 (1989)). The nucleic acid molecule can then be cloned and amplified to obtain sequences that encode the antibody (or antigen-binding domain) of the desired specificity. Phage display technology offers another technique for selecting antibodies that bind to tumor associated antigens, fragments, derivatives or analogs thereof. (See, e.g., International Patent Publications WO 91/17271 and WO 92/01047; Huse et al., supra.)

According to another aspect of the invention, techniques described for the production of single chain antibodies (see, e.g., U.S. Pat. Nos. 4,946,778 and 5,969,108) can be used. An additional aspect of the invention utilizes the techniques described for the construction of a Fab expression library (see, e.g., Huse et al., supra) to allow rapid and easy identification of monoclonal Fab fragments with the desired specificity for tumor associated antigens, fragments, derivatives, or analogs thereof.

Antibody fragments that contain the idiotype of the molecule can be generated by known techniques. For example, such fragments include but are not limited to, F(ab′)₂ fragments, Fab′ fragments, Fab fragments, and Fv fragments. Recombinant Fv fragments can also be produced in eukaryotic cells using, for example, the methods described in U.S. Pat. No. 5,965,405 (the disclosure of which is incorporated by reference herein).

In another embodiment, bi-specific antibodies are provided. Bi-specific antibodies can be monoclonal antibodies that have binding specificities for at least two different antigens. For example, one of the binding specificities can be for a tumor associated antigen and the other one is for another antigen. Alternatively, one specificity is for a first tumor associated antigen, while the other specificity is for a second, different tumor associated antigen.

Methods for making bi-specific antibodies are known in the art. Traditionally, the recombinant production of bi-specific antibodies is based on the co-expression of two immunoglobulin heavy-chain/light-chain pairs, where the two heavy chains have different specificities (see, e.g., Milstein and Cuello, Nature 305:537-39 (1983), the disclosure of which is incorporated by reference herein). Because of the random assortment of immunoglobulin heavy and light chains, these hybridomas (quadromas) produce a potential mixture of ten different antibody molecules, of which some have the desired bi-specific structure. The purification of the desired molecule(s) is usually accomplished by affinity chromatography steps. Similar procedures are disclosed in PCT Patent Publication WO 93/08829, and in Traunecker et al. (EMBO J. 10:3655-59 (1991)) (the disclosures of which are incorporated by reference herein).

Antibody variable domains with the desired binding specificities (antibody-antigen combining sites) can be fused to immunoglobulin constant domain sequences. The fusion typically is with an immunoglobulin heavy-chain constant domain, comprising at least part of the hinge, CH2, and CH3 regions. The first heavy-chain constant region (CH1) containing the site necessary for light-chain binding is usually present in at least one of the fusions. DNAs encoding the immunoglobulin heavy-chain fusions and, if desired, the immunoglobulin light chain, are inserted into separate expression vectors, and are co-transfected into a suitable host organism. For further details of generating bi-specific antibodies see, for example, Suresh et al. (Methods in Enzymology 121:210 (1986), the disclosure of which is incorporated by reference herein).

In the production of antibodies, screening for the desired antibody can be accomplished by techniques known in the art (e.g., ELISA (enzyme-linked immunosorbent assay)). (See, e.g., Harlow and Lane, supra.)

Diagnostics

Methods and compositions for diagnosis of hyperproliferative disease and/or autoimmune disease are also provided. Such methods and compositions can be used to detect, prognose, diagnose, or monitor hyperproliferative disease or autoimmune disease associated with aberrant changes in tumor associated antigen expression, activity and/or immunogenicity. Tumor associated antigen polypeptides (including fragments, derivatives, and analogs thereof), tumor associated antigen nucleic acids (and sequences complementary thereto), and antibodies to tumor associated antigens have uses in diagnostics to detect, prognose, diagnose, or monitor hyperproliferative disease or autoimmune disease. Such hyperproliferative diseases include, but are not limited to, epithelial cancers, such as ovarian cancer, breast cancer, lung cancer, colorectal cancer, and the like. As will be appreciated by the skilled artisan, although the following discussion exemplifies method and compositions for use in the diagnosis, detection, prognosis, or monitoring of hyperproliferative disease, such assays can also be used to diagnose, detect, prognose, or monitor autoimmune disease. The autoimmune disease can be, for example, rheumatoid arthritis, graft versus host disease, systemic lupus erythromatosis (SLE), scleroderma, multiple sclerosis, diabetes, organ rejection, inflammatory bowel disease, psoriasis, and the like.

In one aspect, immunoassays are used to detect antibodies in a subject (“autoimmune antibodies” or “autoantibodies”) to one or more of the tumor associated antigens. For example, immunoassays can be used to detect autoimmune antibodies to at least one of Topoisomerase II alpha, Werner helicase interacting protein, HEXIM1, HDCMA, Deadbox protein-5, and/or Kinesin-like 6 in a sample from a subject. The immunoassays can also be used to detect autoimmune antibodies to at least one of p53, NY-ESO-1, Ubiquilin-1, and/or HOX-B6. In a related embodiment, immunoassays can be used to detected autoimmune antibodies to at least one of Topoisomerase II alpha, Ubiquilin-1, Werner helicase interacting protein, p53 and NY-ESO-1, and optionally tumor associated antigen CA125. In another embodiment, immunoassays can be used to detect autoimmune antibodies to at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, immunoassays can be used to detect autoimmune antibodies to at least one of Topoisomerase II alpha, Werner helicase interacting protein, p53, NY-ESO-1, and optionally, Ubiquilin-1 and/or CA125.

In a further embodiment, immunoassays can be used to detect autoimmune antibodies to at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

The presence of autoimmune antibodies to one or more of these tumor associated antigen can be used as an indication of, or correlated with, a hyperproliferative disease in the subject. In this context, “correlated with” refers to a statistical likelihood that a subject having autoimmune antibodies to one or more tumor associated antigens has a hyperproliferative disease (e.g., an epithelial cancer). Such a correlation can be, for example, an indication that further testing, surgical intervention, and the like, is desired or recommended.

Immunoassays which can be used to detect such autoimmune antibodies include, for example, competitive and non-competitive assay systems such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. (See, e.g., Harlow and Lane, Using Antibodies, A Laboratory Manual, Cold Spring Harbor Laboratory, New York (1999).)

An immunoassay can be carried out, for example, by contacting a subject sample, having antibodies, with at least one tumor associated antigen polypeptide under conditions such that immunospecific binding (complex formation) can occur, and detecting or measuring the amount of any immunospecific binding of antibody to the tumor associated antigen. The tumor associated antigen can be used to detect the presence (e.g., high, low or absent) of antibody to at least one tumor associated antigens in a sample, such as blood, serum, ascites fluid, mucosal fluid (e.g., cervical fluids), and the like, from a subject.

For example, autoimmune antibodies in a subject's sample can be detected by the following method. The tumor associated antigen (or a fragment, derivative and/or analog thereof) is immobilized on a matrix. Then, a sample to be assayed (e.g., blood, serum, ascites fluid, mucosal fluid, and the like) is added and allowed to react at a temperature suitable for immunospecific binding (e.g., from about 4° C. to about 40° C.).

Following the binding reaction (e.g., complex formation), the matrix is washed, and a secondary antibody can be added to the reaction mixture; the secondary antibody (e.g., anti-human antibodies) typically immunospecifically binds to the subject's antibodies. The secondary antibody is allowed to react with autoimmune antibodies bound to the tumor associated antigen on the matrix.

The secondary antibody optionally can be labeled with, for example, a fluorescent substance, a chromogenic substance, a chemiluminescent substance, an enzyme, a radioisotope, by biotinyl moieties, and the like. Examples of detectable labels include, but are not limited to, the following: radioisotopes (e.g., ³H, ¹⁴C, ³²P, ³⁵S, ¹²⁵I, ¹³¹I, and the like), fluorescent molecules (e.g., fluorescein isothiocyanate (FITC), rhodamine, phycoerythrin (PE), phycocyanin, allophycocyanin, ortho-phthaldehyde, fluorescamine, peridinin-chlorophyll a (PerCP), Cy3 (indocarbocyanine), Cy5 (indodicarbocyanine), lanthanide phosphors, and the like), enzymes (e.g., horseradish peroxidase, β-galactosidase, luciferase, alkaline phosphatase), biotinyl groups, and the like. In certain embodiments, detectable labels are attached by spacer arms of various lengths to reduce potential steric hindrance.

The reaction mixture can be washed, as necessary, to remove unbound secondary antibody, and the secondary antibody bound to the matrix can be detected. For example, bound, labeled secondary antibody can be detected by standard colorimetric, radioactive, photometric and/or fluorescent detection means. Detection reagents can be used, if needed. For fluorescent labels, signal can be detected by, for example, a scanning confocal microscope in photon counting mode. Suitable scanning devices are described by, for example, U.S. Pat. Nos. 5,578,832 and 5,631,734 (both incorporated by reference herein). For antibodies labeled with biotin, the reaction can be treated with the appropriate streptavidin-conjugate (e.g., streptavidin-horseradish peroxidase, streptavidin-alkaline phosphatase, streptavidin-luciferase, and the like) and with the appropriate reagents for colorimetric or photometric detection. For radiolabeled antibody, signal can be detected using a scintillation counter, phosphoimager or similar device. Alternatively, the secondary antibody can be unlabeled, and the presence of autoimmune antibodies to a tumor associated antigen can be detected using a labeled tertiary antibody.

Any suitable matrix can be used for immobilizing the tumor associated antigen. For example, for ELISA, the tumor associated antigen can be immobilized on ELISA plates, microtiter plates, and the like. In one embodiment, histidine-tagged tumor associated antigen is bound to H is Sorb ELISA plates (Qiagen). Alternatively, the tumor associated antigen can be immobilized in a sandwich assay.

Autoimmune antibody can also be detected in a conventional Western blotting or dot blotting assay, such as by immobilizing at least one tumor associated antigen to a solid support matrix, such as, for example, nitrocellulose membrane, nylon membrane, PVDF membrane, and the like. The blot can be probed with antibodies for the subject, followed by detection of bound antibody using, for example, a secondary antibody.

The tumor associated antigens can also be immobilized on other matrices. The matrices can have virtually any possible structural configuration so long as the immobilized antigen is capable of being bound by antibody to that antigen. Thus, the support configuration can be spherical, as in a bead, or cylindrical, as in the inside surface of a test tube, or the external surface of a rod. Alternatively, the surface can be flat such as a sheet, test strip, and the like.

Suitable matrices include, for example, gel beads (e.g., Sepharose 4B, Sepharose 6B (Pharmacia Fine Chemicals (Sweden))), dextran gel (e.g., Sephadex G-75, Sephadex G-100, Sephadex G-200 (Pharmacia Fine Chemicals (Sweden))), polyacrylamide gel (e.g., Bio-Gel P-30, Bio-Gel P-60, Bio-Gel P-100 (Bio-Rad Laboratories USA)), cellulose beads (e.g., Avicel (Asahi Chemical Industry Co. Ltd.)), ion exchange cellulose (e.g., diethylaminoethylcellulose, carboxymethylcellulose), physical adsorbents (e.g., glass (glass beads, glass rods, aminoalkyl glass beads, aminoalkyl glass rods)), silicone flakes, styrenic resin (e.g., polystyrene beads, polystyrene particles), immunoassay plates (e.g., Nunc (Denmark)), ion exchange resin (e.g., weakly acidic cation exchange resin (e.g., Amberlite IRC-5 (Rohm & Haas Company (U.S.A.)), Zeo-Karb 226 (Permutit (West Germany)), and weakly basic anion exchange resin (e.g., Amberlite IR-4B, Dowex 3 (Dow Chemical (U.S.A.))), and the like.

Immunoassays to detect autoimmune antibody in a subject sample can also be performed, for example, by contacting a subject sample with a labeled tumor associated antigen polypeptide under conditions such that immunospecific binding can occur (e.g., antibody-tumor associated antigen complex formation), and detecting or measuring the amount of immunospecific complex formation. Such immunoassays can include, for example, immunoprecipitations and RIA's.

Tumor associated antigen can be labeled with, for example, a fluorescent substance, a chromogenic substance, a chemiluminescent substance, an enzyme, a radioisotope, by biotinyl moieties, and the like, as described supra.

Diagnostic assays can also be performed to qualitatively or quantitatively detect tumor associated antigen in a subject's sample. For example, immunoassays can be used to detect at the presence of at least one of the following tumor associated antigens in a subject's sample: Topoisomerase II alpha, Werner helicase interacting protein, HEXIM1, FLJ20267, Deadbox protein-5, and/or Kinesin-like 6. The immunoassays can also be used to detect the presence of at least one of p53, NY-ESO-1 and/or CA125 in a sample from a subject.

The immunoassays can also be used to detect the presence of at least one of p53, NY-ESO-1, Ubiquilin-1, and/or HOX-B6. In a related embodiment, immunoassays can be used to detect the presence of at least one of Topoisomerase II alpha, Ubiquilin-1, Werner helicase interacting protein, p53 and NY-ESO-1, and optionally tumor associated antigen CA125. In another embodiment, immunoassays can be used to detect the presence of at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, immunoassays can be used to detect the presence of at least one of Topoisomerase II alpha, Werner helicase interacting protein, p53, NY-ESO-1, and optionally, Ubiquilin-1 and/or CA125.

In a further embodiment, immunoassays can be used to detect the presence of at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

For example, immunoassays to detect tumor associated antigen can be carried out by a method comprising contacting a sample derived from a subject with an antibodies to tumor associated antigen under conditions such that immunospecific binding (e.g., antibody-tumor associated antigen complex formation) can occur, and detecting or measuring the amount of immunospecific binding. In a specific aspect, binding of antibody to tissue sections from a subject can be used to detect aberrant (e.g., high, low or absent) levels of tumor associated antigen and/or aberrant tumor associated antigen localization. By “aberrant levels” is meant increased or decreased levels or immunogenicity relative to that present, or a standard level representing that present, in an analogous sample from a portion of the body or from a subject not having the hyperproliferative disease.

In a specific embodiment, antibody to tumor associated antigen can be used to assay a subject's tissue, serum or other biological sample for the presence of tumor associated antigen, where an aberrant level or immunogenicity of the tumor associated antigen is an indication of a hyperproliferative disease (e.g., an epithelial cancer) or is correlated with the presence of hyperproliferative disease in a subject. The immunoassays which can be used to detect tumor associated antigen include, for example, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. (See, e.g., Harlow and Lane, Using Antibodies, A Laboratory Manual, Cold Spring Harbor Laboratory, New York (1999).)

For example, antibodies can be used to quantitatively or qualitatively detect the presence of tumor associated antigens using immunofluorescence techniques employing a fluorescently labeled antibody (see, e.g., supra) coupled with light microscopic, flow cytometric, or fluorimetric detection. Such techniques can be used to detect tumor associated antigens that are expressed on the cell surface. Thus, the techniques described herein can be used to detect specific cells, within a population of cells, having altered tumor associated antigen expression or immunogenicity.

Immunoassays can also be employed histologically, as in immunofluorescence or immunoelectron microscopy, for in situ detection of tumor associated antigen. In situ detection can be accomplished by removing a histological sample from a subject, and contacting the sample with a labeled antibody. The antibody is typically contacted with the sample by overlaying the labeled antibody onto the sample. Through the use of such a procedure, the presence of the tumor associated antigen can be determined and/or the distribution of the antigen in the histological sample can be examined. Those of ordinary skill in the art will readily appreciate that any of a wide variety of histological methods (such as staining procedures) can be modified in order to achieve such in situ detection.

In certain embodiments, a biological sample from a subject is contacted with and immobilized onto a matrix, such as, for example, nitrocellulose, or other solid support (see supra) which is capable of immobilizing cells, cell particles or polypeptides. The matrix can then be washed with suitable buffers followed by treatment with the labeled antibody. The matrix can then be washed, as needed, with the buffer to remove unbound antibody. The amount of bound label on the matrix can be detected by conventional means.

Bound, labeled antibody can be detected, for example, by standard colorimetric, radioactive, photometric and/or fluorescent detection means. Detection reagents can be used, if needed. For fluorescent labels, signals can be detected by, for example, a scanning confocal microscope in photon counting mode. Appropriate scanning devices are described by, for example, U.S. Pat. Nos. 5,578,832 and 5,631,734 (both incorporated by reference herein). For antibodies labeled with biotin, the reaction can be treated with the appropriate streptavidin-conjugate (e.g., streptavidin-horseradish peroxidase, streptavidin-alkaline phosphatase, streptavidin-luciferase, and the like) and then treated with the appropriate reagents for calorimetric or photometric detection. For radiolabeled antibody, signals can be detected using a scintillation counter, phosphoimager or similar device.

In another aspect, diagnostic assays are provided to detect the altered expression of tumor associated antigen genes. In this context, “altered expression” refers to increased or decreased levels of RNA expression from a gene relative to that present, or a standard level representing that present, in an analogous sample from a portion of the body or from a subject not having the hyperproliferative disease. Tumor associated antigen nucleic acid sequences, or fragments thereof comprising about at least 8, at least 15 or at least 30 nucleotides can be used as hybridization probes. Hybridization assays can be used to detect, prognose, diagnose, or monitor hyperproliferative disease associated with altered expression of tumor associated antigen genes. In particular, such a hybridization assay can be carried out by a method comprising contacting a sample having nucleic acids (target nucleic acids) with a nucleic acid probe capable of hybridizing to tumor associated antigen nucleic acid, under conditions such that hybridization can occur, and detecting or measuring any resulting hybridization.

In specific embodiments, hyperproliferative disease can be diagnosed, or its suspected presence can be screened for, or a predisposition to develop such disease can be detected, by detecting tumor associated antigen RNA associated with altered expression of the tumor associated antigen gene. Altered expression of a tumor associated antigen gene can also be correlated with the presence of hyperproliferative disease in a subject. Suitable hybridization assays include, for example, Northern blots, dot blots, RT-PCR, quantitative PCR, and the like.

In a specific embodiment, levels of tumor associated antigen mRNA are detected or measured, in which increased levels indicate that the subject has, or a predisposition to developing, a hyperproliferative disease, or where increased expression is correlated with the presence of hyperproliferative disease in a subject.

Diagnostic procedures can also be performed in situ directly upon, for example, tissue sections (e.g., fixed and/or frozen) of subject tissue obtained from biopsies or resections, such that no nucleic acid purification is necessary. Tumor associated antigens nucleic acids can be used as probes and/or primers for such in situ procedures (see, e.g., Nuovo, PCR In Situ Hybridization: Protocols and Applications, Raven Press, NY (1992), the disclosure of which is incorporated by reference herein).

Diagnostic methods for the detection of tumor associated antigen nucleic acids can also involve, for example, contacting (e.g., incubating) nucleic acids from a subject's sample with one or more labeled nucleic acids, under conditions favorable for the specific annealing of the nucleic acids to their complementary sequences. Typically, the lengths of these nucleic acid reagents are at least 15 to 30 nucleotides. After incubation, non-annealed nucleic acids are removed. The presence of bound nucleic acids from the sample, if any such molecules exist, is then detected. Using such a detection scheme, the nucleic acid from the tissue or cell type of interest can be immobilized, for example, to a solid support such as a membrane, or a plastic surface such as that on a microtiter plate or polystyrene beads.

Nucleic acid arrays can be used to monitor altered expression of one or more tumor associated genes, such as, for example, Topoisomerase II alpha, Werner helicase interacting protein, HEXIM1, HDCMA, Deadbox protein-5, and/or Kinesin-like 6. Nucleic acid arrays can further be used to detect altered expression of one or more of genes encoding p53, NY-ESO-1, Ubiquilin-1 and/or HOX-B6.

In additional embodiments, nucleic acid arrays can also be used to detect altered expression of genes encoding at least one of p53, NY-ESO-1, Ubiquilin-1, and/or HOX-B6. In a related embodiment, nucleic acid arrays can also be used to detect altered expression of genes encoding at least one of Topoisomerase II alpha, Ubiquilin-1, Werner helicase interacting protein, p53 and NY-ESO-1, and optionally tumor associated antigen CA125. In another embodiment, nucleic acid arrays can also be used to detect altered expression of genes encoding at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, nucleic acid arrays can also be used to detect altered expression of genes encoding at least one of Topoisomerase II alpha, Werner helicase interacting protein, p53, NY-ESO-1, and optionally, Ubiquilin-1 and/or CA125.

In a further embodiment, nucleic acid arrays can also be used to detect altered expression of genes encoding at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

In yet another embodiment, nucleic acid arrays can be used to detect altered expression of genes encoding the following: TOP2a and DDX5; or TOP2a, DDX5 and HOXB6; or TOP2a, DDX5, HOXB6 and NY-ESO-1; or TOP2a, DDX5, HOXB6, NY-ESO-1 and HER2/neu and HEXIM1; or TOP2a, DDX5, HOXB6, NY-ESO-1, HER2/neu and HEXIM1, BRCA1 and HOXB7.

Typically, an array of polynucleotide probes can be contacted with a sample of target nucleic acids to produce a hybridization pattern. The binding of the target nucleic acids to one or more probes of the array can be detected to obtain a qualitative and/or quantitative profile of expression of the tumor associated antigen gene.

An array of polynucleotide probes stably associated with the surface of a substantially planar solid support is typically contacted with a sample of target nucleic acids under hybridization conditions sufficient to produce a hybridization pattern of complementary probe/target complexes. A variety of different arrays can be used and are known in the art. The probe molecules of the arrays can be polynucleotides or hybridizing derivatives or analogs thereof, including: nucleic acids in which the phosphodiester linkage has been replaced with a substitute linkage, such as phophorothioate, methylimino, methyl-phosphonate, phosphoramidate, guanidine, and the like; nucleic acids in which the ribose subunit has been substituted, for example, hexose phosphodiester; peptide nucleic acids; and the like. The length of the probes will generally range from about 10 to about 1000 nucleotides. In some embodiments the probes will be oligonucleotides and usually range from about 15 to about 150 nucleotides and more usually from about 15 to about 100 nucleotides in length. In other embodiments the probes can be longer, usually ranging in length from about 150 to about 1000 nucleotides. The probes can be single or double stranded, usually single stranded, and can be PCR fragments amplified from cDNA. The probe molecules on the surface of the substrates can typically correspond to at least one of the tumor associated antigen genes and can be positioned on the array at a known locations so that positive hybridization events can be correlated to expression of a particular gene in the physiological source from which the target nucleic acid sample is derived. Because of the manner in which the target nucleic acid sample can be generated, as described below, the arrays of probes can have sequences that are complementary to the non-template strands of the gene to which they correspond.

The substrates with which the probe molecules are stably associated can be fabricated from a variety of materials, including plastics, ceramics, metals, gels, membranes, glasses, and the like. The arrays can be produced according to any convenient methodology, such as preforming the probes and then stably associating them with the surface of the support or growing the probes directly on the support. A number of different array configurations and methods for their production are known to those of skill in the art and disclosed in, for example, U.S. patent numbers: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; the disclosures of which are herein incorporated by reference.

The target nucleic acid is typically contacted with the array under conditions sufficient for hybridization of target nucleic acid to probe to occur. Suitable hybridization conditions are well known to those of skill in the art and discussed in, for example, Sambrook et al. (supra) and PCT Patent Publication WO 95/21944 (incorporated by reference herein). For example, low stringency hybridization conditions can be at 50° C. and 6×SSC while hybridization under stringent conditions can be at 50° C. or higher and 1×SSC.

In one embodiment, the amount of tumor associated antigen nucleic acids in the sample can be quantitated. (See, e.g., U.S. Pat. No. 6,004,755, the disclosure of which is incorporated by reference herein.) For example, the target nucleic acids in the sample can be end-labeled in a manner such that each of the target nucleic acids in the sample produces a signal of the same specific activity. By generating the same specific activity is meant that each individual target polynucleotide in the sample being assayed is labeled in a manner such that the molecule is capable of providing the same signal (e.g., the same intensity of signal) as every other labeled target in the sample. Each of the target nucleic acids generates a signal of the same specific activity because the number of labeled nucleotide bases in each of the target molecules is either identical or substantially the same.

The label can be capable of providing a detectable signal, either directly or through interaction with one or more additional members of a signal producing system. Labels that are directly detectable and that can find use in the subject invention include, for example, fluorescent labels. The fluorescers of interest include fluorescers in which the wavelength of light absorbed by the fluorescer will generally range from about 300 to 900 nm, usually from about 400 to 800 nm. The absorbance maximum will typically occur at a wavelength ranging from about 500 to 800 nm. Specific fluorescers of interest for use in singly labeled primers include, for example, fluorescein, rhodamine, BODIPY, cyanine dyes and the like, and are further described in Smith et al (Nature 321:647-79 (1986)). Suitable radioactive isotopes include, for example, ³⁵S, ³²P, ³H, etc. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. Capture moieties of interest include ligands, such as, for example, biotin where the other member of the signal producing system could be fluorescently labeled streptavidin, and the like.

In certain applications, it can be desired to analyze populations of target nucleic acids from two or more samples. Such samples can be differentially labeled. Alternatively, targets nucleic acids from different samples are separately contacted to identical probe arrays under conditions of hybridization, typically stringent hybridization conditions, such that labeled nucleic acids hybridize to their complementary probes on the substrate surface, and the target nucleic acids bound to the array separately detected. A set of standard nucleic acid molecules can optionally be used. For example, the standard nucleic acids can be provided by reverse transcribing standard RNA.

Following hybridization, a washing step can be employed to remove non-specifically bound nucleic acid from the support surface, generating a pattern of hybridized nucleic acid on the substrate surface. A variety of wash solutions and protocols for their use are known to those of skill in the art and can be used.

Where the label on the target nucleic acid is not directly detectable, the array can be contacted with the other member(s) of the signal producing system that is being employed. For example, where the label on the target is biotin, the array can be contacted with streptavidin-fluorescer conjugate under conditions sufficient for binding between the specific binding member pairs to occur. Following contact, unbound members of the signal producing system can be removed (e.g., by washing). The specific wash conditions employed can depend on the specific nature of the signal producing system that is employed, and are known to those of skill in the art familiar with the particular signal producing system employed.

The resultant hybridization pattern(s) of target nucleic acids bound to the array can be visualized or detected in a variety of ways, with the particular manner of detection being chosen based on the particular label of the nucleic acid. For example, detection means can include scintillation counting, autoradiography, fluorescence measurement, calorimetric measurement, light emission measurement, and the like.

Prior to detection or visualization, the array of hybridized target/probe complexes can be optionally treated with an endonuclease. The endonuclease degrades single stranded, but not double stranded DNA. A variety of different endonucleases are known and can be used. Such nucleases include, for example, mung bean nuclease, S1 nuclease, and the like.

Following detection or visualization, the hybridization pattern can be used to determine qualitative and/or quantitative information about the expression of tumor associated antigen genes. The hybridization patterns of different samples can be compared with each other, and/or with a control sample, to identify differences between the patterns. The hybridization arrays can also be used to identify differential gene expression, in the analysis of diseased and normal tissue (e.g., neoplastic and normal tissue), different tissue or subtissue types; and the like.

In another aspect, the methylation profile of one or more tumor associated antigen genes can be determined qualitatively and/or quantitatively. Changes in methylation can be associated with altered expression, either increased or decreased expression, of the gene(s). As used herein, a “methylation profile” refers to the presence or absence of at least one methylated nucleic acid residue in a tumor associated antigen gene.

In one aspect, the tumor associated antigen gene can encode Topoisomerase II alpha, Werner helicase interacting protein, HEXIM1, HDCMA, Deadbox protein-5, or Kinesin-like 6. The tumor associated antigen gene can encode at least one of p53, NY-ESO-1, Ubiquilin-1, and/or HOX-B6. In a related embodiment, tumor associated antigen gene can encode at least one of Topoisomerase II alpha, Ubiquilin-1, Werner helicase interacting protein, p53 and NY-ESO-1, and optionally tumor associated antigen CA125. In another embodiment, the tumor associated antigen gene can encode at least one of Topoisomerase II alpha, Werner helicase interacting protein, HEXIM1, HDCMA, Deadbox protein-5, Ubiquilin-1, HOX-B6, p53 and NY-ESO-1. In yet another embodiment, the tumor associated antigen gene can encode at least one of Topoisomerase II alpha, Werner helicase interacting protein, p53, NY-ESO-1, and optionally, Ubiquilin-1 and/or CA125.

In a further embodiment, the tumor associated antigen gene can encode at least one of Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

In an exemplary embodiment, the methylation profile of a tumor associated gene can be determined by: obtaining a nucleic acid-containing sample (e.g., containing genomic DNA) from a subject, determining a methylation profile for a tumor associated antigen gene in the sample; comparing the methylation profile of the tumor associated antigen gene with a known methylation profile for the tumor associated antigen gene; and prognosing or diagnosing a risk of hyperproliferative disease in the subject. The methylation profile can be determined, for example, by contacting the nucleic acid-containing sample with an agent that modifies unmethylated cytosine and amplifying the nucleic acid in the sample. The amplified nucleic acids can be examined to determine the methylation profile of the tumor associated gene. The nucleic acid can be amplified with primers that hybridize with a tumor associated antigen gene sequence (e.g., random primers or primers based on a portion of the gene sequence). In certain embodiments, the primers can distinguish between modified methylated and non-methylated nucleic acid. The methylated nucleic acid in the sample can be detected based on the presence or absence of amplification products produced in the amplification step. The amplifying step can be, for example, polymerase chain reaction, ligase chain reaction, use of Qβ replicase, and the like. The modifying agent can be, for example, bisulfite.

The methylation profile of any suitable portion of a tumor associated antigen gene can be determined. For example, the methylation profile of the promoter region, coding region, intronic regions, and/or 3′ noncoding region can be determined. In an exemplary embodiment, oligonucleotide primers can be used to amplify the promoter region of the tumor associated antigen gene following contacting with the modifying agent.

Methods for determining the methylation profile of a gene include, for example, restriction digestion using methylation-sensitive and/or methylation dependent restriction enzymes, methylation specific PCR, restriction digestion of PCR products amplified from bisulfite-converted DNA, bisulfite genomic sequencing, COBRA, DNA methylation fingerprinting, and Ms-SNuPe. Suitable methods for detecting methylation profiles, and changes in such profiles, are disclosed by, for example, Herman et al. (Proc. Natl. Acad. Sci. USA 95:6870-75 (1998); Proc. Natl. Acad. Sci. USA 93:9821-26 (1992)); Deng et al. (Nucleic Acids Res. 30:E13 (2002)); Gonzalgo and Jones (Nucleic Acids Res. 25:2529-31 (1997)); Worm et al. (Clin. Chem. 47:1183-09 (2001)); Xiong and Laird (Nucleic Acids Res. 25:2532-34 (1997)); U.S. Pat. Nos. 6,017,704; 6,214,556; and 6,331,393; the disclosures of which are incorporated by reference herein.)

Kits for diagnostic use are also provided, that comprise in one or more containers a tumor associated antigen, and, optionally, antibody to the tumor associated antigen. The tumor associated antigen can optionally be labeled (e.g., with a detectable marker, such as, for example, a chemiluminescent, enzymatic, fluorescent, and/or radioactive moiety). For example, a kit can include Topoisomerase II alpha, Werner helicase interacting protein, HEXIM1, HDCMA, Deadbox protein-5, and/or Kinesin-like 6. In addition, a kit can optionally include one or more of p53, NY-ESO-1, Ubiquilin-1 and/or HOX-B6.

In additional embodiments, a kit can include p53, NY-ESO-1, Ubiquilin-1, and/or HOX-B6. In a related embodiment, a kit can include Topoisomerase II alpha, Ubiquilin-1, Werner helicase interacting protein, p53 and NY-ESO-1, and optionally tumor associated antigen CA125. In another embodiment, a kits can include Topoisomerase II alpha, Werner helicase interacting protein, HEXIM1, HDCMA, Deadbox protein-5, Ubiquilin-1, HOX-B6, p53 and NY-ESO-1. In yet another embodiment, a kit can include Topoisomerase II alpha, Werner helicase interacting protein, p53, NY-ESO-1, and optionally, Ubiquilin-1 and/or CA125.

In a further embodiment, a kit can include Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

Kits for diagnostic use are also provided that comprise in one or more containers antibody to tumor associated antigen antibody, and, optionally, a labeled binding partner to the antibody. Alternatively, the antibody to the tumor associated antigen can be labeled (with a detectable marker, such as, for example, a chemiluminescent, enzymatic, fluorescent, and/or radioactive moiety).

In certain embodiments, a kit can include antibody to Topoisomerase II alpha, Werner helicase interacting protein, HEXIM1, HDCMA, Deadbox protein-5, and/or Kinesin-like 6. The kit can also include antibody to at least one of p53, NY-ESO-1, Ubiquilin-1 and/or HOX-B6.

In additional embodiments, a kit can include antibody to p53, NY-ESO-1, Ubiquilin-1, and/or HOX-B6. In a related embodiment, a kit can include antibody to Topoisomerase II alpha, Ubiquilin-1, Werner helicase interacting protein, p53 and NY-ESO-1, and optionally tumor associated antigen CA125. In another embodiment, a kit can include antibody to Topoisomerase II alpha, Werner helicase interacting protein, HEXIM1, HDCMA, Deadbox protein-5, Ubiquilin-1, HOX-B6, p53 and NY-ESO-1. In yet another embodiment, a kit can include antibody to Topoisomerase II alpha, Werner helicase interacting protein, p53, NY-ESO-1, and optionally, Ubiquilin-1 and/or CA125.

In a further embodiment, a kit can include antibody to Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

A kit is also provided that comprises in one or more containers a nucleic acid probe capable of hybridizing to tumor associated antigen RNA. In a specific embodiment, a kit can comprise in one or more containers a pair of primers (e.g., each in the size range of 6-30 nucleotides, or more in length) that are capable of priming amplification (e.g., by polymerase chain reaction (see, e.g., Innis et al., PCR Protocols, Academic Press, Inc., San Diego, Calif. (1990)), ligase chain reaction (see, e.g. EP 320,308), use of Qβ replicase, cyclic probe reaction, or other methods known in the art under appropriate reaction conditions, of at least a portion of a tumor associated antigen nucleic acid. A kit can optionally further comprise in a container a predetermined amount of at least one purified tumor associated antigen or nucleic acid, for example, for use as a standard or control.

A kit can include nucleic acid probes capable of hybridizing to tumor associated antigen RNA for Topoisomerase II alpha, Werner helicase interacting protein, HEXIM1, HDCMA, Deadbox protein-5, and/or Kinesin-like 6. A kit can optionally further include nucleic acid probes capable of hybridizing to tumor associated antigen RNA for p53, NY-ESO-1, Ubiquilin-1 and/or HOX-B6.

In additional embodiments, a kit can include nucleic acid probes capable of hybridizing to tumor associated antigen RNA for p53, NY-ESO-1, Ubiquilin-1, and/or HOX-B6. In a related embodiment, a kit can include nucleic acid probes capable of hybridizing to tumor associated antigen RNA for Topoisomerase II alpha, Ubiquilin-1, Werner helicase interacting protein, p53 and NY-ESO-1, and optionally tumor associated antigen CA125. In another embodiment, a kit can include nucleic acid probes capable of hybridizing to tumor associated antigen RNA for Topoisomerase II alpha, Werner helicase interacting protein, HEXIM1, HDCMA, Deadbox protein-5, Ubiquilin-1, HOX-B6, p53 and NY-ESO-1. In yet another embodiment, a kit can include nucleic acid probes capable of hybridizing to tumor associated antigen RNA for Topoisomerase II alpha, Werner helicase interacting protein, p53, NY-ESO-1, and optionally, Ubiquilin-1 and/or CA125.

In a further embodiment, a kit can include nucleic acid probes capable of hybridizing to tumor associated antigen RNA corresponding to for Topoisomerase II alpha, Werner helicase interacting protein, HEXIM1, HDCMA, Deadbox protein-5, and/or Kinesin-like 6, optionally at least one of p53, NY-ESO-1 and CA125, and at least one of ZFP161, Ubiquilin-1, HOX-B6, IFI27, YB-1, KIAA0136, Osteonectin, F-box only protein 21, and ILF3.

In yet another embodiment, a kit can include nucleic acid probes capable of hybridizing to tumor associated antigen RNA for the following: TOP2a and DDX5; or TOP2a, DDX5 and HOXB6; or TOP2a, DDX5, HOXB6 and NY-ESO-1; or TOP2a, DDX5, HOXB6, NY-ESO-1 and HER2/neu and HEXIM1; or TOP2a, DDX5, HOXB6, NY-ESO-1, HER2/neu and HEXIM1, BRCA1 and HOXB7.

Treatment

In another aspect according to the present invention, compositions and method of using such compositions for the treatment of hyperproliferative disease, such as by immunotherapy are also provided. As used herein, immunotherapy can be passive or active. Passive immunotherapy as defined herein is the passive transfer of antibody to a subject (e.g., patient). Active immunization includes the induction of antibody and/or T-cell or other lymphocyte or lymphocyte-derived biomolecule responses in a subject (e.g., a patient). For example, active induction of an immune response can be the result of providing the subject with an antigen to which antibodies are produced by the subject. As appreciated by one of ordinary skill in the art, the antigen can be provided by injecting a tumor associated antigen polypeptide into a subject, or contacting the subject with a nucleic acid capable of expressing the tumor associated antigen and under conditions for expression of the antigen.

As will be appreciated by the skilled artisan, the expression pattern of a tumor associated antigen across will dictate which hyperproliferative diseases can be treated using that antigen. Furthermore, to be therapeutically useful, an tumor associated antigen is typically expressed at significantly higher levels in the target cell type (e.g., an epithelial cancer) than in normal tissues (with the exception of immune-privileged sites such as the eye, or dispensible tissues such as ovary or testis).

Immunotherapeutic compositions according to the present invention can include purified forms of at least one tumor associated antigens for use as vaccines. One or more tumor associated antigens can be introduced into subjects in a manner designed to boost the endogenous immune response to that antigen. The tumor associated antigen can be delivered, for example, as whole protein, smaller peptides, or DNA or RNA molecules encoding all or a portion of the antigen. An adjuvant can be included with the tumor associated antigen to help boost the immune response. In an exemplary vaccination strategy, the tumor associated antigen, or an antigenic fragment thereof, can be loaded onto subject-derived antigen presenting cells (e.g., dendritic cells) which are then introduced into the subject. In another exemplary strategy, the tumor associated antigen, or an antigenic fragment thereof, can be loaded onto multimeric complexes, such as, for example, multimeric MHC Class I or MHC Class II complexes, which are then introduced into the subject. (See, e.g., U.S. Pat. No. 5,635,363, U.S. patent application Ser. No. 10/116,846; the disclosures of which are incorporated by reference herein.)

The tumor associated antigens can comprise at least an immunogenic portion of a tumor associated antigen polypeptide, or a derivative or analog thereof, as described herein. Tumor associated antigen polypeptides can be of any suitable length. Additional sequences derived from the native tumor associated antigen protein and/or heterologous sequences can be present, and such sequences can (but need not) possess further immunogenic or antigenic properties.

An “immunogenic portion,” as used herein is a portion of an antigen that is recognized (i.e., specifically bound) by a B-cell and/or T-cell surface antigen receptor. Such immunogenic portions generally comprise at least 5 amino acid residues, more typically at least 10 or at least 20 amino acid residues of a tumor associated antigen or a derivative or analog thereof. Further immunogenic portions can generally be identified using well known techniques, such as those summarized in Paul, Fundamental Immunology (3rd ed., pp. 243-247 (Raven Press, 1993)) and references cited therein. Such techniques include screening polypeptides for the ability to react with tumor associated antigen-specific antibodies, antisera and/or T-cell lines or clones. As used herein, antisera and antibodies are “tumor associated antigen-specific” if they specifically bind to a tumor associated antigen (i.e., they react with the tumor associated antigen in an ELISA or other immunoassay, and do not react detectably with unrelated proteins). Such antisera, antibodies and T cells can be prepared as described herein, and using well known techniques. An immunogenic portion of a native tumor associated antigen is a portion that reacts with such antisera, antibodies and/or T-cells at a level that is not substantially less than the reactivity of the full length polypeptide (e.g., in an ELISA and/or T-cell reactivity assay). Such immunogenic portions can react within such assays at a level that is similar to or greater than the reactivity of the full length protein. Such screens can generally be performed using methods well known to those of ordinary skill in the art, such as those described in Harlow and Lane, Antibodies: A Laboratory Manual (Cold Spring Harbor Laboratory, 1988). For example, a tumor associated antigen polypeptide can be immobilized on a solid support and contacted with subject's sera to allow binding of antibodies within the sera to the immobilized polypeptide. Unbound sera can then be removed and bound antibodies detected using, for example, ¹²⁵I-labeled Protein A.

Tumor associated antigen polypeptides can be prepared using any of a variety of well known techniques. For example, recombinant polypeptides encoded by DNA sequences can be readily prepared from the DNA sequences using any of a variety of expression vectors known to those of ordinary skill in the art. Expression can be achieved in any appropriate host cell that has been transformed or transfected with an expression vector containing a DNA molecule that encodes a recombinant polypeptide. Suitable host cells include prokaryotes, yeast and higher eukaryotic cells. Typically, the host cells employed are E. coli, yeast or a mammalian cell line such as COS or CHO. Supernatants from suitable host/vector systems which secrete recombinant protein or polypeptide into culture media optionally can be concentrated using a commercially available filter. Following concentration, the concentrate can be applied to a suitable purification matrix such as an affinity matrix or an ion exchange resin. Finally, one or more reverse phase HPLC steps can be employed to further purify a recombinant polypeptide.

Fragments or other variants (e.g., derivatives or analogs) typically having fewer than about 100 amino acids, and generally fewer than about 50 amino acids, can also be generated by synthetic means, using techniques well known to those of ordinary skill in the art. For example, such polypeptides can be synthesized using any of the commercially available solid-phase techniques, such as the Merrifield solid-phase synthesis method, where amino acids are sequentially added to a growing amino acid chain. (See, e.g., Merrifield, J. Am. Chem. Soc. 85:2149-46 (1963).) Equipment for automated synthesis of polypeptides is commercially available from suppliers such as Applied BioSystems, Inc. (Foster City, Calif.), and can be operated according to the manufacturer's instructions.

Within certain specific embodiments, a tumor associated antigen polypeptide can be a fusion protein that comprises multiple polypeptides, or that comprises a fusion partner and a tumor associated antigen or a variant of such a protein. A fusion partner can, for example, assist in providing T helper epitopes (an immunological fusion partner), typically T helper epitopes recognized by humans, or can assist in expressing the protein (an expression enhancer) at higher yields than the native recombinant protein. Certain fusion partners are both immunological and expression enhancing fusion partners. Other fusion partners can be selected so as to increase the solubility of the protein or to enable the protein to be targeted to desired intracellular compartments. Still further fusion partners include affinity tags, which facilitate purification of the protein.

Fusion proteins can generally be prepared using standard techniques, including chemical conjugation. Typically, a fusion protein is expressed as a recombinant protein, allowing the production of increased levels, relative to a non-fused protein, in an expression system. Briefly, DNA sequences encoding the polypeptide components can be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence can be employed to separate first and the second polypeptide components of a fusion protein by a distance sufficient to ensure that each polypeptide folds into its secondary and tertiary structures. Such a peptide linker sequence can be incorporated into a fusion protein using standard techniques well known in the art. Suitable peptide linker sequences can be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Typical peptide linker sequences contain Gly, Asn and Ser residues. Other near neutral amino acids, such as Thr and Ala can also be used in the linker sequence. Amino acid sequences which can be usefully employed as linkers include those disclosed in Maratea et al. (Gene 40:39-46 (1985)), Murphy et al. (Proc. Natl. Acad. Sci. USA 83:8258-62 (1986)), U.S. Pat. No. 4,935,233 and U.S. Pat. No. 4,751,180. The linker sequence can generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptide. Similarly, stop codons can be included to end translation and transcription termination signals are present 3′ to the DNA sequence encoding the second polypeptide.

Fusion proteins are also provided that comprise a polypeptide according to the present invention together with an unrelated immunogenic protein. Typically, the immunogenic protein is capable of eliciting a recall response. Examples of such proteins include tetanus, tuberculosis and hepatitis proteins (see, e.g., Stoute et al., New Engl. J. Med. 336:86-91 (1997)).

In another aspect, the immunotherapeutic composition can comprise antibodies to at least one to tumor associated antigen. For example, humanized antibodies to one or more tumor associated antigens can be administered to a human subject. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (see Jones et al., Nature 321:522-25 (1986); Riechmann et al., Nature 332:323-29 (1988); Presta, Curr. OP. Struct. Biol. 2:593-96 (1992)).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. For example, humanization can be performed by substituting rodent CDRs or CDR sequences for the corresponding sequences of a human antibody. (See, e.g., Jones et al., Nature 321:522-25 (1986); Riechmann et al., Nature 332:323-27 (1988); Verhoeyen et al., Science 239:1534-36 (1988)). Accordingly, such humanized antibodies are chimeric antibodies (see, e.g., U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985); Boerner et al., J. Immunol. 147:86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-59 (1994); Morrison, Nature 368:812-13 (1994); Fishwild et al., Nature Biotechnology 14:845-51 (1996); Neuberger, Nature Biotechnology 14:826 (1996); and Lonberg and Huszar, Intern. Rev. Immunol. 13:65-93 (1995).

Immunotherapeutic compositions can also, or alternatively, comprise T cells specific for one or more tumor associated antigens. Such cells can be prepared in vitro or ex vivo, using standard procedures. For example, T cells can be present within (or isolated from) bone marrow, peripheral blood or a fraction of bone marrow or peripheral blood of a mammal, such as a patient, using a commercially available cell separation system, such as, for example, CEPRATE™ system, available from CellPro Inc., Bothell Wash. (see also U.S. Pat. No. 5,240,856; U.S. Pat. No. 5,215,926; WO 89/06280; WO 91/16116 and WO 92/07243). Alternatively, T cells can be derived from related or unrelated humans, non-human animals, cell lines or cultures.

T cells can be stimulated with one or more tumor associated antigens polynucleotides encoding an tumor associated antigen and/or antigen presenting cells (APCs) that expresses such an antigen. Such stimulation can be performed under conditions and for a time sufficient to permit the generation of T cells that are specific for the antigen. Typically, the tumor associated antigen or polynucleotide is present within a delivery vehicle, such as a microsphere, to facilitate the generation of specific T cells.

T cells are considered to be specific for a tumor associated antigen if the T cells kill target cells coated with a tumor associated antigen polypeptide or expressing a gene encoding such a polypeptide. T cell specificity can be evaluated using any of a variety of standard techniques. For example, within a chromium release assay or proliferation assay, a stimulation index of more than two fold increase in lysis and/or proliferation, compared to negative controls, indicates T cell specificity. Such assays can be performed, for example, as described in Chen et al., Cancer Res. 54:1065-70 (1994). Alternatively, detection of the proliferation of T cells can be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor associated antigen (e.g., about 200 ng/ml-about 100 μg/ml, typically about 100 ng/ml-about 25 μg/ml) for 3-7 days typically results in at least a two fold increase in proliferation of the T cells and/or contact for about 2-3 hours can result in activation of the T cells, as measured using standard cytokine assays in which a two fold increase in the level of cytokine release (e.g., TNF or IFN-gamma) is indicative of T cell activation (see, e.g., Coligan et al., Current Protocols in Immunology, vol. 1, Wiley Interscience (1998)). T cells that have been activated in response to a tumor associated antigen polynucleotide or tumor associated antigen-expressing APC can be CD4⁺ and/or CD8⁺. Tumor associated antigen-specific T cells can be expanded using standard techniques. The T cells are derived from a subject or a related or unrelated donor and can administered to the subject following stimulation and expansion.

For therapeutic purposes, CD4⁺ or CD8⁺ T cells that proliferate in response to a tumor associated antigen polynucleotide or APC can be expanded in number either in vitro or in vivo. Proliferation of such T cells in vitro can be accomplished in a variety of ways. For example, the T cells can be re-exposed to a tumor associated antigen, with or without the addition of T cell growth factors, such as interleukin-2, and/or stimulator cells that synthesize the tumor associated antigen. Alternatively, one or more T cells that proliferate in the presence of the tumor associated antigen can be expanded in number by cloning. Methods for cloning cells are well known in the art, and include limiting dilution. Following expansion, the cells can be administered back to the subject as described, for example, by Chang et al. (Crit. Rev. Oncol. Hematol. 22:213 (1996)).

In a specific embodiment, adoptive T cell therapy is used, in which the tumor associated antigen is used for in vitro stimulation of lymphocytes from a subject so as to induce the outgrowth of CD4⁺ and/or CD8⁺ T cells that recognize that antigen. Such T cells can then be propagated in vitro either as oligoclonal lines, or as monoclonal cultures. T cells can be expanded to great numbers with repeated exposure to antigen and mitogenic cytokines such as interleukin-2 and -15. T cells can then be re-infused into the subject, where they are expected to mount a curative immune response to tumor cells expressing the appropriate antigen.

Within certain aspects, tumor associated antigen polypeptides, polynucleotides, antibodies and/or immune system cells as described herein can be incorporated into pharmaceutical compositions or vaccines. Pharmaceutical compositions comprise one or more such compounds or cells and a physiologically acceptable carrier. Vaccines can comprise one or more such compounds or cells and a non-specific immune response enhancer. A non-specific immune response enhancer can be any substance that enhances an immune response to an exogenous antigen. Examples of non-specific immune response enhancers include adjuvants, biodegradable microspheres (e.g., polylactic galactide) and liposomes (into which the compound is incorporated; see, e.g., U.S. Pat. No. 4,235,877). Vaccine preparation is generally described in, for example, Powell and Newman, eds., “Vaccine Design (the subunit and adjuvant approach),” Plenum Press (New York, 1995). Pharmaceutical compositions and vaccines within the scope of the present invention can also include other compounds, which can be biologically active or inactive. For example, one or more immunogenic portions of other tumor associated antigens can be present, either incorporated into a fusion polypeptide or as a separate compound within the composition or vaccine.

A pharmaceutical composition or vaccine can include DNA encoding one or more of the tumor associated antigen polypeptides, as described above, such that the polypeptide is generated in situ. As noted above, the DNA can be present within any of a variety of delivery systems known to those of ordinary skill in the art, including nucleic acid expression systems, bacteria and viral expression systems. Appropriate nucleic acid expression systems can include the necessary DNA sequences for expression in the subject (e.g., a suitable promoter and terminating signal). Bacterial delivery systems can involve the administration of a bacterium (such as Bacillus-Calmette-Guerrin) that expresses an immunogenic portion of a polypeptide on its cell surface. In a typically embodiment, the DNA can be introduced using a viral expression system (e.g., vaccinia or other pox virus, retrovirus, or adenovirus), which can involve the use of a non-pathogenic (defective), replication competent virus. Suitable systems are disclosed, for example, in Fisher-Hoch et al. (Proc. Natl. Acad. Sci. USA 86:317-21 (1989)); Flexner et al. (Ann. N.Y. Acad. Sci. 569:86-103 (1989)), Flexner et al. (Vaccine 8:17-21 (1990)), U.S. Pat. Nos. 4,603,112, 4,769,330 and 5,017,487, WO 89/01973, U.S. Pat. No. 4,777,127; GB 2,200,651, EP 0,345,242, WO 91/02805, Berkner (Biotechniques 6:616-27 (1988)), Rosenfeld et al. (Science 252:431-34 (1991)), Kolls et al. (Proc. Natl. Acad. Sci. USA 91:215-19 (1994)), Kass-Eisler et al., Proc. Natl. Acad. Sci. USA 90:11498-502 (1993)), Guzman et al. (Circulation 88:2838-48 (1993)), and Guzman et al. (Cir. Res. 73:1202-07 (1993)). Techniques for incorporating DNA into such expression systems are well known to those of ordinary skill in the art. The DNA can also be “naked,” as described, for example, in Ulmer et al. (Science 259:1745-49 (1993)) and reviewed by Cohen (Science 259:1691-92 (1993)). The uptake of naked DNA can be increased by coating the DNA onto biodegradable beads, which are efficiently transported into the cells.

While any suitable carrier known to those of ordinary skill in the art can be employed in the pharmaceutical compositions of this invention, the type of carrier will vary depending on the mode of administration. Compositions according to the present invention can be formulated for any appropriate manner of administration, including for example, topical, oral, nasal, intravenous, intracranial, intraperitoneal, subcutaneous or intramuscular administration. For parenteral administration, such as subcutaneous injection, the carrier typically comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, can be employed. Biodegradable microspheres (e.g., polylactate polyglycolate) can also be employed as carriers for the pharmaceutical compositions. Suitable biodegradable microspheres are disclosed, for example, in U.S. Pat. Nos. 4,897,268 and 5,075,109.

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention can be formulated as a lyophilizate. Compounds can also be encapsulated within liposomes using well known technology.

Any of a variety of non-specific immune response enhancers can be employed. For example, an adjuvant can be included. Most adjuvants contain a substance designed to protect the antigen from rapid catabolism, such as aluminum hydroxide or mineral oil, and a stimulator of immune responses, such as lipid A, Bortadella pertussis- or Mycobacterium tuberculosis-derived proteins. Suitable adjuvants are commercially available as, for example, Freund's Incomplete Adjuvant and Complete Adjuvant (Difco Laboratories, Detroit, Mich.), Merck Adjuvant 65 (Merck and Company, Inc., Rahway, N.J.), alum, biodegradable microspheres, monophosphoryl lipid A and quil A. Cytokines, such as GM-CSF or interleukin-2, -7, or -12, can also be used as adjuvants.

Within the vaccines provided herein, the adjuvant composition can be typically designed to induce an immune response predominantly of the Th1 type. High levels of Th1-type cytokines (e.g., IFN-gamma, IL-2 and IL-12) tend to favor the induction of cell mediated immune responses to an administered antigen. In contrast, high levels of Th2-type cytokines (e.g., IL-4, IL-5, IL-6, IL-10 and TNF-beta) tend to favor the induction of humoral immune responses. Following application of a vaccine as provided herein, a subject will support an immune response that includes Th1- and Th2-type responses. Within an embodiment, in which a response is predominantly Th1-type, the level of Th1-type cytokines will increase to a greater extent than the level of Th2-type cytokines. The levels of these cytokines can be readily assessed using standard assays. For a review of the families of cytokines, see, for example, Mosmann and Coffman (Ann. Rev. Immunol. 7:145-73 (1989)).

Suitable adjuvants for use in eliciting a predominantly Th1-type response include, for example, a combination of monophosphoryl lipid A, typically 3-de-O-acylated monophosphoryl lipid A (3D-MPL), together with an aluminum salt. MPL adjuvants are available from Ribi ImmunoChem Research Inc. (Hamilton, Mont.; see U.S. Pat. Nos. 4,436,727; 4,877,611; 4,866,034 and 4,912,094). Another adjuvant is AS-2 (SmithKline Beecham). CpG-containing oligonucleotides (in which the CpG dinucleotide is unmethylated) can also induce a predominantly Th1 response. Such oligonucleotides are well known and are described, for example, in WO 96/02555. Another adjuvant is a saponin, typically QS21, which can be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other formulations can comprise an oil-in-water emulsion and tocopherol. A particularly potent adjuvant formulation involving QS21, 3D-MPL and tocopherol in an oil-in-water emulsion is described in WO 95/17210. Any vaccine provided herein can be prepared using well known methods that result in a combination of antigen, immune response enhancer and a suitable carrier or excipient.

The compositions described herein can be administered as part of a sustained release formulation (i.e., a formulation such as a capsule or sponge that effects a slow release of compound following administration). Such formulations can generally be prepared using well known technology and administered by, for example, oral, rectal or subcutaneous implantation, or by implantation at the desired target site. Sustained-release formulations can contain a polypeptide, polynucleotide or antibody dispersed in a carrier matrix and/or contained within a reservoir surrounded by a rate controlling membrane. Carriers for use within such formulations are biocompatible, and can also be biodegradable; typically the formulation provides a relatively constant level of active component release. The amount of active compound contained within a sustained release formulation depends upon the site of implantation, the rate and expected duration of release and the nature of the condition to be treated or prevented.

Any of a variety of delivery vehicles can be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that can be engineered to be efficient APCs. Such cells can, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs can generally be isolated from any of a variety of biological fluids and organs, including tumor and peritumoral tissues, and can be autologous, allogeneic, syngeneic or xenogeneic cells.

Certain embodiments can use dendritic cells or progenitors thereof as antigen-presenting cells. Dendritic cells are highly potent APCs (Banchereau and Steinman, Nature 392:245-51 (1998)) and have been shown to be effective as a physiological adjuvant for eliciting prophylactic or therapeutic antitumor immunity (see Timmerman and Levy, Ann. Rev. Med. 50:507-29 (1999)). In general, dendritic cells can be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells can be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) can be used within a vaccine (see Zitvogel et al., Nature Med. 4:594-600 (1998)).

Dendritic cells and progenitors can be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells can be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow can be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature’ cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fc-gamma receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86).

APCs can generally be transfected with a polynucleotide encoding a tumor associated antigen (or portion or other variant thereof) such that the antigen, or an immunogenic portion thereof, is expressed on the cell surface. Such transfection can take place ex vivo, and a composition or vaccine comprising such transfected cells can then be used for therapeutic purposes, as described herein. Alternatively, a gene delivery vehicle that targets a dendritic or other antigen presenting cell can be administered to a subject, resulting in transfection that occurs in vivo. In vivo and ex vivo transfection of dendritic cells, for example, can generally be performed using any methods known in the art, such as those described in WO 97/24447, or the gene gun approach described by Mahvi et al. (Immunology and cell Biology 75:456-60 (1997)). Antigen loading of dendritic cells can be achieved, for example, by incubating dendritic cells or progenitor cells with the tumor associated antigen polypeptide, DNA (naked or within a plasmid vector) or RNA; or with tumor associated antigen-expressing recombinant bacterium or viruses (e.g., vaccinia, fowlpox, adenovirus or lentivirus vectors). Prior to loading, a tumor associated antigen can be covalently conjugated to an immunological partner that provides T cell help (e.g. a carrier molecule). Alternatively, a dendritic cell may be pulsed with a non-conjugated immunological partner, separately or in the presence of the polypeptide.

In further aspects, the compositions described herein can be used for immunotherapy of hyperproliferative disease, such as ovarian cancer. Within such methods, pharmaceutical compositions and vaccines are typically administered to a subject, such as a patient. As used herein, a “patient” refers to any warm-blooded animal, typically a human. A patient may or may not be afflicted with hyperproliferative disease. Accordingly, the above pharmaceutical compositions and vaccines can be used to prevent the development of a hyperproliferative disease or to treat a patient afflicted with a hyperproliferative disease. Within certain embodiments, a patient is afflicted with ovarian cancer. Pharmaceutical compositions and vaccines can be administered prior to or following surgical removal of primary tumors and/or treatment such as administration of radiotherapy or conventional chemotherapeutic drugs.

Routes and frequency of administration, as well as dosage, will vary from individual to individual, and can be readily established using standard techniques. In general, the pharmaceutical compositions and vaccines can be administered by injection (e.g., intracutaneous, intramuscular, intravenous or subcutaneous), intranasally (e.g., by aspiration), orally or in the bed of a resected tumor. Typically, between 1 and 10 doses can be administered over a 52 week period. In certain embodiments, 6 doses can be administered, at intervals of 1 month, and booster vaccinations can be given periodically thereafter. Alternate protocols can be appropriate for individual subjects.

A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a subject or by vaccine-dependent generation of cytolytic effector cells capable of killing the subject's tumor cells in vitro. Such vaccines are also be capable of causing an immune response that leads to an improved clinical outcome (e.g., more frequent remissions, complete or partial or longer disease-free survival) in vaccinated subjects as compared to non-vaccinated subjects. In general, for pharmaceutical compositions and vaccines comprising one or more polypeptides, the amount of each polypeptide present in a dose ranges from about 100 μg to 5 mg per kg of host. Suitable dose sizes will vary with the size of the subject, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor associated antigen generally correlate with an improved clinical outcome. Such immune responses can generally be evaluated using standard proliferation, cytotoxicity or cytokine assays, which can be performed using samples obtained from a subject before and after treatment.

The following examples are provided merely as illustrative of various aspects of the invention and shall not be construed to limit the invention in any way.

### EXAMPLES

### Example 1

The following example describes a protocol for SEREX immuoscreening to identify of tumor associated antigens using serum from patients having ovarian cancer.

SEREX Immunoscreening

Three cDNA expression libraries were constructed for SEREX screening using RNA from ten stage III/IV serous ovarian tumors, an ovarian tumor cell line H3907, and normal human testis. Poly-A was selected from each source using an mRNA Separator kit from Clontech or an Oligotex mRNA kit from Qiagen. The RNA from the ten stage III/IV serous ovarian tumors was pooled. The selected mRNA was converted to cDNA with a modified ZAP cDNA synthesis kit (Stratagene) and cloned into lambda TriplEx (Clontech, for ovarian tumor derived RNA) or lambda UniZap ZR (Stratagene; for testis and H3907 derived RNA). Prior to screening, serum from stage III ovarian cancer patients was pre-cleared of E. coli specific antibodies using an E. coli affinity resin (5Prime3Prime) according to the manufacturers instructions.

SEREX immunoscreening was performed essentially as described by Tureci et al. (Hybridoma 18:23-28 (1999); Mol. Med. Today 3:342-49 (1997), both incorporated by reference herein) or Sahin et al. (Proc. Natl. Acad. Sci. USA 92:11810-813 (1995); incorporated by reference herein). Briefly, aliquots of the expanded library were plated at 2×10³ PFU/100 mm plate, overlaid with IPTG impregnated nitrocellulose membranes and incubated overnight at 37° C. The following morning, lifts were washed three times in Tris buffered saline (TBS: 20 mM Tris-HCl pH 7.5 and 150 mM NaCl)+0.05% Tween 20, blocked in TBS+1% BSA for 2 hours and exposed to serum diluted 1:200 in TBS/BSA overnight at room temperature. Lifts were washed 3 times in TBS and incubated with an alkaline phosphatase-linked goat anti-human IgG secondary antibody for 45 minutes at room temp. After three washes in TBS, lifts were developed in nitro blue tetrazolium chloride/5-bromo-4-chloro-3-indoyl phosphate (NBT/BCIP) for approximately 5 minutes, stopped in water for 20 minutes and dried. Positive phage plaques were picked and stored in SM buffer (100 mM NaCl, 50 mM Tris-HCl pH 7.5 and 10 mM MgSO₄) at 4° C. with a drop of chloroform.

SEREX Array Analysis

To evaluate antibody responses to multiple antigens with a large number of sera, an array-based secondary screening method was developed. Standard 98 well sized rectangular NZY petri dishes were coated with NZY top agar containing non-infected Y1090-host bacteria. A multi-channel repeating pipettor was then used to spot 0.7 μl of purified antigen-encoding phage suspension in defined positions in a 6×8 array. Each phage was spotted in duplicate in non-adjacent positions. Positive controls of phage encoding human IgG cDNA, and negative control phage of empty parental lambda phage DNA were included in duplicate on each array. This technique allows construction of multiple identical array plates. Following phage application, plates were incubated for 4 hours at 37° C.

IPTG impregnated rectangular nitrocellulose membranes were then placed on the surface of each plate and incubation was continued overnight. Array membranes were removed, washed and exposed to individual patient or normal control sera as described for standard SEREX screening. Detection was carried out using an alkaline phosphatase-linked human IgG-specific secondary antibody, followed by standard NBT/BCIP development. Membranes were visually scored by comparing the staining intensity centered over the candidate antigen plaques with the signals generated on the positive and negative control plaques included on each array. Staining in clear excess of the negative controls was scored as positive. Phage scoring positive with one or more patient sera and no control sera were classified as candidate ovarian tumor antigens.

Phage encoding candidate tumor antigens were purified by re-plating and re-screening at lower phage titers. Purified lambda TriplEx phage were converted to plasmids by infecting Cre expressing hosts (BM25.8) according to the manufacturers protocol. Lambda Zap clones were converted by co-infection with ExAssist helper phage, harvesting the supernatant and re-infecting the SOLR strain provided by Stratagene. Miniprep DNA was isolated for each clone and sequencing was carried out using ABI BigDye sequencing reagents and T7 and T3 primers.

Sequencing and Analysis

Sequencing templates were prepared using QIAprep mini spin columns according to the manufacturer's instructions. Both ends of clone all clones were sequenced using the following vector primers; TCCGAGATCTGGACGAGC (sense primer) (SEQ ID NO:1) and TAATACGACTCACTATAGGG (anti-sense primer) (SEQ ID NO:2). Sequences were analyzed using BLASTn searches against NCBI (http://www.ncbi.nlm.nih.gov/) nr, EST and Unigene databases.

Production of Antigens for ELISA

Full-length coding sequences for NY-ESO-1, p53, UBQLN1, HDCMA, HEXIM1, MAGE family proteins, SSX1 and RUVBL were amplified from IMAGE clones and ligated in-frame into His-tag expression vectors pcDNA4/HISMax (p53, NY-ESO-1, HDCMA, MAGEA4, MAGEB2) or pQE (UBQLN1, HEXIM1, RUVBL, MAGEA1, MAGEA3/6, SSX1). The coding regions were checked by DNA sequence analysis. pQE constructs were transfected into M15 hosts and expression was induced with 1 mM IPTG. Antigens expressed in bacteria were purified under denaturing conditions on Talon metal-affinity resin according to the manufacturers instructions (Clontech). Purification was confirmed by western blot and silver-stained SDS-PAGE analysis.

pcDNA/HISMax constructs were transiently transfected into COS7 cells with Lipofectamine Plus (Invitrogen). Cells were incubated for three days, then trypsinized, washed in PBS and lysed in PBS, 0.6% NP-40 and a protease inhibitor cocktail (Boehringer), quick frozen and stored at −80° C. Expression of his-tagged antigens in COS7 cells was confirmed by western blot of whole cell lysates using a His-tag-specific monoclonal antibody (Penta-his, Qiagen).

ELISA Protocol

Serum antibodies to tumor associated antigens are detected by ELISA. 96 well Immulon ELISA plates were coated overnight with 50 μl 0.75 μg/ml His-tag specific monoclonal antibody (Qiagen catalog number 34660) in carbonate buffer. Plates were blocked with 150 μl PBS/0.2% Tween-20/1% non-fat milk for 2 hours, washed 3× with PBS/Tween-20 and coated with 50 μl of purified antigens (25 μg/ml) or COS7 lysates (1 mg/ml) in PBS/Tween/milk. Plates were held overnight at 4° C. Following 2 washes with PBS/Tween, 50 μl of serum diluted 1:100 in blocking buffer was added to each well. Following overnight incubation with serum, plates were washed 4× with PBS/Tween and exposed to 50 μl of HRP-linked anti-human IgG secondary antibody (Amersham NA933) diluted 1:5000 in blocking buffer for 45 minutes at room temperature. Plates were washed 8× with PBS/Tween and developed with 75 μl TMB reagent (Kirkegaard and Perry Laboratories) for 5 minutes and stopped with 75 μl HCl. Plates were read at 405 nm.

Each sera was tested in triplicate against both SEREX-derived antigen and a second non-antigen his-tag fusion protein (purified GAPDH for antigens purified from bacteria, and LacZ for antigens produced in eukaryotic cells) as a specificity control. Serum used for cloning each antigen was included on each plate as a positive control. A titration of human IgG from 150 to 0 ng/ml was also included on each plate as an additional control. Twenty-five sera from patients with late-stage serous ovarian cancer and twenty sera from cancer free women over age 30 were analyzed for each antigen. A positive score was determined as an antigen-specific value greater than the mean+3 standard deviations of the normal controls. All positive sera were titered from 1:50 to 1:800 to verify that the autoantibody response also diminished proportionately and therefore was specific for the antigen under consideration.

### Example 2

The SEREX immunoscreening methodology (supra) was used to identify tumor antigens associated with human ovarian tumors. Screening with panels of sera from ovarian cancer patients has identified a set of antigens that are immunogenic in more than one patient, and not in a matched panel of 20 normal control serum donors.

Serex Screening

SEREX immuno-screening was conducted to identify ovarian tumor antigens. SEREX screens were conducted in an allogeneic manner, in which the sera used for screening were unrelated to the tumors used for library construction. Initially, two cDNA libraries were constructed and screened, one derived from RNA pooled from ten stage III/IV serous ovarian tumors, and the other from the ovarian tumor cell line H3907. Each library contained more that 1×10⁶ primary clones and had average insert sizes of 1.5 kb or greater. Both libraries were screened with sera from 25 late stage serous ovarian cancer patients drawn at diagnosis or immediately prior to surgery (serum panel #1). To allow for extensive yet efficient screening, pairs of serum samples were pooled, and each serum pool was then exposed to a minimum of 1.5×10⁵ clones from each library. Plaques expressing immunoreactive proteins were picked, plaque purified and tested for binding to secondary antibody alone. Those that bound the secondary antibody alone were classified as false positives and discarded. Rescue and sequencing of a subset of these clones confirmed that they were library-derived clones of human IgG. The remaining immunoreactive clones were classified as candidate ovarian tumor antigens and were sequenced. In total, primary SEREX screening of the H3907 and tumor-derived cDNA libraries with 13 pairs of patient-derived sera yielded a set of 18 candidate ovarian tumor antigens (Table 1).

Up to 40 identical arrays were constructed, with each position on the array corresponding to a phage encoding a defined candidate antigen. Each array was exposed to a single serum from either an ovarian cancer patient (from serum panel #1) or a cancer-free normal control. The control sera were from women over age 30 with no personal history of cancer or autoimmune disease. Of the 18 antigens tested, 13 antigens were recognized only by the patient serum sample with which they were originally cloned (Table 1, H3907 and tumor derived antigens). However, five antigens bound serum antibodies from more than one cancer patient versus 0 out of 20 cancer-free controls. These included NY-ESO-1, p53, TOP2A, RUVBL and UBQLN1. Altogether, 10 out of 25 sera from serum panel #1 recognized one or more of these 5 antigens, suggesting that serum antibody responses are relatively common in ovarian cancer, and that additional SEREX screening was required to expand the panel of relevant antigens.

To identify additional antigens, serum pools from patients who scored negative for autoantibodies to the above antigens were used for further screening of both the H3907 and the tumor-derived libraries. Despite screening to an average depth of 5×10⁵ plaques, no additional antigens were identified, initially suggesting that this subset of ovarian cancer patients may not have mounted a tumor-specific autoantibody response. However, the two libraries used for primary SEREX screening yielded different sets of antigens, even when screened with serum from the same patients (Table 1). The H3907 library yielded the known ovarian cancer tumor antigen p53, as well as the novel candidates TOP2A, RUVBL, Nucleolar coiled body phosphoprotein 1 (NOLC1), Chromosome condensation protein-G (HCAPG), Dihydrolipoamide dehydrogenase (DLD), Deadbox polypeptide-9 (DDX9), Stathmin 1 (STMN1) and Interleukin enhancer binding factor 3 (ILF3). The tumor-derived library yielded NY-ESO-1, HOX-B6, UBQLN1, Zinc finger protein 161 (ZFP161), HEXIM1, Osteonectin, CD44 antigen, Y-box binding factor 1 (YB-1), and F-box only protein 21 (FBXO21).

TABLE 1

| | | | | Patient | Patient | Control |
|---|---|---|---|---|---|---|
| | | | | serum | serum | serum |
| | | Antigen | Gene | panel # 1 | panel # 2 | panel |
| cDNA Library | Antigen | Abbreviation | Name | N = 25 | N = 25 | N = 20 |
| H3907 | p53 | p53 | TP53 | 2 | 4 | 0 |
| H3907 | Topoisomerase II a | TOP2A | TOP2A | 2 | 1 | 0 |
| H3907 | Putative helicase | RUVBL | WHIP | 2 | 0 | 0 |
| | RUVBL | | | | | |
| H3907 | Nucleolar | NOLC1 | NOLC1 | 1 | 0 | 0 |
| | phosphoprotein 1 | | | | | |
| H3907 | Chromosome | | | | | |
| | condensation | | | | | |
| protein G | HCAPG | HCAP-G | 1 | 0 | 0 | |
| H3907 | Dibydrolipoamide | | | | | |
| dehydrogenase | DLD | DLD | 1 | 0 | 0 | |
| H3907 | Deadbox protein 9 | DDX9 | DDX9 | 1 | 0 | 0 |
| H3907 | Stathmin 1 | STMN1 | STMN1 | 1 | 0 | 0 |
| H3907 | Interleukin enhancer | | | | | |
| binding factor 3 | ILF3 | ILF3 | 1 | 0 | 0 | |
| tumor | NY-ESO-1 | NY-ESO-1 | CTAG1 | 5 | 5 | 0 |
| tumor | Ubiquilin-1 | UBQLN1 | UBQLN1 | 2 | 1 | 0 |
| tumor | Homeobox B6 | HOXB6 | HOXB6 | 1 | 1 | 0 |
| tumor | Zinc finger protein | ZFP161 | ZFP161 | 1 | 0 | 0 |
| | 161 | | | | | |
| tumor | HMBA inducible | HEXIM1 | HIS1 | 1 | 1 | 0 |
| tumor | Osteonectin | SPARC | SPARC | 1 | 0 | 0 |
| tumor | CD44 antigen | CD44 | CD44 | 1 | 0 | 0 |
| tumor | Y-Box binding | YB-1 | YB-1 | 1 | 0 | 0 |
| | factor-1 | | | | | |
| tumor | F-box only protein 21 | FBX21 | FBXO21 | 1 | 0 | 0 |
| testes | HDCMA18P protein | | | | | |
| (FLJ20267) | HDCMA | HDCMA18P | 2 | 1 | 0 | |
| testes | Deadbox polypeptide 5 | DDX5 | DDX5 | 1 | 0 | 0 |
| testes | FLJ22318 | FLJ22318 | FLJ22318 | 1 | 0 | 0 |

To identify additional tumor antigens not represented in the H3907 and tumor-derived libraries, a third cDNA library was screened. To increase the likelihood of detecting rare or abnormally expressed antigens, the third library was constructed using mRNA isolated from normal human testes. Testes tissue is known to express a wide variety of mRNA species, including a class of tumor antigens that are expressed exclusively in normal testes, fetal tissue and tumors (Cancer Testes, or CT antigens, see Sahn et al., Proc. Natl. Acad. Sci. USA 92:11810-13 (1995)). Examples of CT antigens include NY-ESO-1 and members of the MAGE gene family.

Primary SEREX screening of the testes library was conducted using sera that had not yielded candidate antigens during previous screening efforts. Approximately 2×10⁵ phage were screened with each of six serum pools (two sera per pool). Three of the previously “antigen negative” serum pools yielded a total of 7 novel candidate antigens (Table 1). Array analysis of these antigens using sera from panel #1 showed that one antigen, DDX5 was bound by serum IgG from two ovarian cancer patients, while the remaining antigens bound antibodies in only a single patient. None of the antigens were recognized by the 20 normal control sera. Interestingly, no additional CT antigens were cloned despite the use of a testes library.

Frequency of Antibody Responses to SEREX-Defined Ovarian Tumor Antigens.

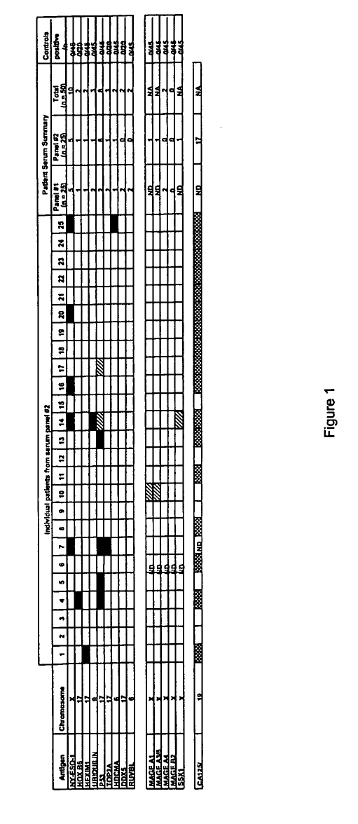

Approximately 50% (13/25) of the patients from serum panel #1 had antibodies to at least one of the 25 SEREX-defined antigens. However, serum panel #1 used the serum samples used for cloning, and does not represent an unbiased estimate of antibody frequency. Therefore, all candidate antigens were exposed to a second panel of sera from 25 patients with late stage serous ovarian cancer (serum panel #2, see Table 1). None of the sera in the second panel had been used for primary SEREX screening. Of these patients, 36% (9/25) demonstrated serum antibodies to at least one antigen in the panel (FIG. 1). Furthermore, when the array results from serum panels #1 and #2 were combined, nine antigens (NY-ESO-1, HOX-B6, HEXIM1, UBQLN1, p53, TOP2A, HDCMA, DDX5 and RUVBL) were recognized by antibodies from two or more patients versus 0/20 controls, suggesting that they were useful as biomarkers for ovarian cancer.

SEREX arrays allow rapid validation of potential ovarian tumor antigens. However, only lambda clones for NY-ESO-1 and HDCMA included the full-length protein coding sequence. Therefore, it is possible that for the other antigens, some patients might express antibodies to epitopes not encoded by the library-derived lambda clones. The SEREX arrays may underestimate the frequency of antibody responses to these antigens. For this reason, specific ELISAs were developed using full-length His-tagged recombinant proteins for the nine best SEREX-derived antigens. ELISAs were successfully developed for NY-ESO-1, p53, HEXIM1, UBQLN1, HDCMA and RUVBL, whereas TOP2A, DDX5 and HOX-B6 were difficult to express as recombinant His-tagged proteins and were not developed further.

ELISAs were performed with patient serum panel #2 and sera from a total of 45 disease-free control serum donors. One patient from panel #2, (serum #6) was unavailable for testing. As a positive control, each ELISA plate also included the serum from panel #1 used to clone each antigen. Each serum was tested in triplicate against both individual SEREX-derived antigens and, as a specificity control, a non-antigen his-tag fusion protein (either human GAPDH or bacterial beta-galactosidase).

ELISAs were repeated twice, and each positive sera was then titered from 1:50 to 1:800. Sera were only scored as positive if the autoantibodies titered appropriately. All ELISA results were in complete agreement with the SEREX array analysis for NY-ESO-1, HEXIM1, UBQLN1, HDCMA and RUVBL. The only exceptions were two patients who scored negative for p53 antibodies by SEREX array, but scored positive by ELISA. These additional patients may express antibodies to peptide epitopes not encoded in the original cDNA clone used in the SEREX arrays. Alternatively, these antibodies may recognize conformational epitopes generated in eukaryotic cells, which was the source of the recombinant p53 used for ELISA. The addition of these two patients brings the total number of patients in serum panel #2 expressing antibodies to at least one SEREX-defined ovarian tumor antigen to 10 out of 25 (40%).

Frequency of Antibody Responses to MAGEA1, A3/6, A4, B2 and SSX1.

Several groups have reported autoantibody responses to various CT antigens in patients with ovarian cancer, yet with the exception of NY-ESO-1 such antigens were not found in the SEREX screens. To investigate why this might be the case, patient sera was tested for autoantibodies to several commonly expressed CT antigens, including MAGEA1, MAGEA3/6, MAGEA4, MAGEB2 and SSX1. ELISAs were performed using serum panel #2 and a total of 45 normal controls. Only two of the 25 patients showed a response to any of these CT antigens. One patient who had previously been found to express autoantibodies to NY-ESO-1, UBQLN1, and p53 also expressed antibodies to SSX1 (FIG. 1). A second patient who was negative for autoantibody responses to the SEREX-defined antigens nevertheless expressed antibodies to MAGEA1 and MAGEA3/6. Thus, autoantibody responses to CT antigens of the MAGE and SSX families appear to be rare in ovarian cancer, which is in general agreement with prior reports and likely explains why such antigens were not found in these SEREX screens. (See Stockert et al., J. Exp. Med. 187:1349-54 (1998).) The addition of the two positive patients brought the overall number of patients expressing autoantibodies to one or more tumor antigens to 11 out of 25 (44%).

Summary

SEREX immunoscreening identified a panel of tumor associated antigens. Immuno-screening of three independent cDNA libraries with sera from 25 late stage ovarian cancer patients identified an initial set of 25 potential antigens (Table 1, FIG. 1). These 25 antigens were evaluated in an array-based SEREX protocol using 50 late-stage ovarian cancer patient sera (serum panels #1 and #2, Table 1) and a panel of normal control sera. Out of 25 identified antigens, nine including NY-ESO-1, HOX-B6, UBQLN1, TOP2A, HEXIM1, HDCMA, p53, DDX5 and RUVBL were recognized by serum antibodies present in multiple ovarian cancer patients and not in normal controls (Table 1 and FIG. 1). Autoantibody responses to this panel of nine antigens were evaluated in an independent set of ovarian cancer sera (serum panel #2), which revealed that 40% (10/25) of patients express antibodies to at least one of these antigens.

The identified tumor associated antigens represent three common classes of tumor antigen: (1) cancer/testes or “CT” antigens, which are expressed exclusively in tumors, fetal tissues and normal testes (e.g., NY-ESO-1); (2) antigens that are expressed at higher levels in tumors compared to normal tissues (e.g., TOP2A, and Deadbox-polypeptide 5 (DDX5)); and (3) antigens with tumor-specific mutations (e.g., p53). The five remaining antigens (HEXIM1, HOX-B6, HDCMA, RUVBL, UBQLN1) are broadly expressed in normal tissue, which is typical of many SEREX-defined antigens.

The four most commonly recognized antigens were NY-ESO-1, p53, TOP2A and UBQLN1. Antibodies to NY-ESO-1 were detected in 20% of all ovarian tumors (10/50) and 0 out of 20 normal controls. p53 was the second most frequently recognized antigen derived from these screens (16%, 8/50). UBQLN1 and TOP2A were the third most frequently recognized antigens (6%, 3/50 each).

By ELISA and SEREX array analysis, approximately 44% (11/25) of patients express antibodies to at least one ovarian tumor antigen in this study. In addition, serum antibodies to other antigens have been described, including Homeobox-B7, HER2/neu and additional members of the MAGE, SSX and heat shock protein (HSP) families. (Stockert et al., J. Exp. Med. 187:1349-54 (1998); Disis et al., Breast Cancer Res. Treat. 62:245-52 (2000); Naora et al., Proc. Natl. Acad. Sci. USA 98:4060-65 (2001); Korneeva et al., Int. J. Cancer 87:824-28 (2000).)

### Example 3

CA125 is currently a clinically applied serum marker for ovarian cancer. To explore potential overlap and complementation between CA125 and autoantibodies to the nine best SEREX-defined antigens, CA125 radioimmuno-assays (RIA) were performed on 24/25 sera from panel #2 (FIG. 1; one sera from panel #2 was not available for testing).

Radioimmunoassay for CA125

CA-125 protein was measured utilizing the Fujirebio Diagnostics, Inc. (formerly Centocor) CA125 II radioimmunoassay (RIA) kits (Fujirebio Diagnostics, Malvern, Pa.). This is a one step “sandwich” RIA that uses the M11 antibody as capture antibody and radioiodinated OC125 antibody as detector antibody. The test was carried out according to the directions supplied with the kits and assay calibration was based on a Fujirebio provided reference CA125 preparation. Assays were run in batches of 40 study samples in duplicate, with duplicate high and low internal and external controls, five levels of concentrations of standards and two blanks in each batch. Inter-assay and intra-assay CV's were <10%.

Comparison of Antibody Responses to Serum CA125 Levels.

17 of 24 patients (70%) from panel #2 had serum CA125 levels greater than the accepted clinical threshold of 35 U/ml. Of the 7 patients with normal CA125 levels, one had antibodies to the SEREX antigen p53 and one had antibodies to MAGEA1 and MAGEA3/6. Thus, although this is a small sample size, it appears that serum autoantibody responses can occur in CA125 negative patients, suggesting they may represent complementary markers.

### Example 4

Chromosomal Locations

The chromosomal locations of genes encoding SEREX-defined antigens were determined using mapping resources provided at the National Center for Biotechnology Information (http://www.ncbi.nlm.nih.gov/genome). The chromosomal locations are listed in FIG. 1. Unexpectedly, four of the nine most frequently recognized antigens are encoded within a broad region of 17q21.1-21.3 that includes the genes for HER2/neu, BRCA1 and HOXB7.

The gene encoding TOP2A clusters (within 1M base) with the gene for HER2/neu at 17q21.1, while a second cluster includes Homeobox protein B6 and the previously identified ovarian tumor antigen Homeobox-B7 at 17q21.32.16. Two other antigens, HEXIM1 and DDX5, are also encoded in this region, within 1M base of HOXB7. DDX5 lies telomeric to the HOXB cluster, while the current mapping information suggests that the gene for HEXIM1 lies between the ERBB2/TOP2A cluster and the HOXB cluster.

ERBB2, TOP2A and DDX5 show elevated expression in multiple ovarian tumors, suggesting that mis-regulation of genes on 17q is a common event in ovarian cancer. These findings represent multiple, functionally unrelated tumor antigens encoded within a single chromosomal region. These data suggest that a common genetic lesion in ovarian tumors may lead to miss-expression and consequent immune recognition of multiple gene products encoded within this region of chromosome 17.

### Example 5

Real Time PCR

RNA from human tissues was either purchased from Clontech, or isolated from tissues collected and provided by the Pacific Ovarian Cancer Research Consortium using RNazol according to the manufacturers instructions. A wide variety of normal human tissues were evaluated, specifically, adrenal gland, bone marrow, brain, cerebellum, heart, kidney, liver, lung, skeletal muscle, placenta, prostate, salivary gland, spleen, testes, trachea, thymus, thyroid, uterus, fetal brain and fetal liver. RNA was reverse transcribed using Invitrogen's Superscript II enzyme according to manufacturer's protocol. cDNAs were then used as templates for real-time PCR using the reporter dye SYBR Green. Primer sequences are shown in Table 2.

TABLE 2

| | | |
|---|---|---|
| Primer | Sequence | SEQ ID NO. |

| | | |
|---|---|---|
| DDX5 sense | ATGACTACCCTAACTCCTCAGAGG | 3 |
| DDX5 antisense | AAGCAGGCTAGAGTAACCTCTGTC | 4 |
| HDCMA sense | CCAACAGGGAAGAGTGTCGCACCC | 5 |
| HDCMA antisense | ACTGCTTGAGCATCCTCAGGAGCT | 6 |
| HER2/neu sense | CTCTGAGACTGATGGCTACGTTGC | 7 |
| HER2/neu antisense | CGTCTTTGACGACCCCATTCTTCC | 8 |
| HEXIM1 sense | CGATGACGACTTCATGGAAGAAGG | 9 |
| HEXIM1 antisense | ACTCCTTGATGAGCTCCTGCTTGCTC | 10 |
| HOXB6 sense | CACTCCGGTCTACCCGTGGATGCA | 11 |
| HOXB6 antisense | CATATCTTGATCTGCCTCTCCGTCAG | 12 |
| HOXB7 sense | CACTCCGGTCTACCCGTGGATGCA | 13 |
| HOXB7 antisense | ATCTGTCTTTCCGTGAGGCAGAGC | 14 |
| MUC16/CA125 sense | GCCACTGGCTCGGAGAGTAGA | 15 |
| MUC16/CA125 antisense | CCGGCAAGTTCCAGTCATTG | 16 |
| NY-ESO-1 sense | GGTGCTTCTGAAGGAGTTCACTGTG | 17 |
| NY-ESO-1 antisense | TTAGCGCCTCTGCCCTGAGGGAGG | 18 |
| p53 sense | CACTGCCCAACAACACCAGCTCCT | 19 |
| p53 antisense | GTCTGAGTCAGGCCCTTCTGTCTTG | 20 |
| RUVBL sense | CTGCGTTAACACAAGCGGTTGCTG | 21 |
| RUVBL antisense | CCCAAATCCTTCATCAGCCTAGTG | 22 |
| TMP21 sense | CGACGCTTCTTCAAGGCCAA | 23 |
| TMP21 antisense | ATGGAAGCCCAAGCTGCTGA | 24 |
| TOP2A sense | GTCAGTCTTCCACCTCCACTACCG | 25 |
| TOP2A antisense | TGCCCGAGGAGCCACAGCTGAGTC | 26 |
| UBQLN1 sense | CAGTTATTCAGCAGATGCTGCAGG | 27 |
| UBQLN1 antisense | GAGCCCAGTAACCTTTCAATAGCTGC | 28 |

PCR was performed in 384-well plates in an ABI7900 Real-time PCR machine under the following conditions: 60 seconds at 94° C., 40 cycles of 25 seconds at 94° C., 25 seconds at 60° C., and 45 seconds at 72° C. using 1 U/μl of Biolase enzyme made by Bioline and 0.12 mM dNTPs, 0.12 mM of each primer, 1.5 mM MgCl₂ and the supplied buffer. The SYBR green emission was recorded several times during each cycle, thus monitoring in real time the accumulation of newly synthesized DNA molecules. Standards on each 384-well plate were used to determine the DNA concentration. The standards were a twofold serial dilution of cDNA made from a white blood cell RNA preparation amplified using the primers for TMP21 (GenBank accession number U61734), a gene that was found to be expressed in all tissues tested so far.

The PCR products were run on a 2% agarose gel in 1×TBE to ensure that the SYBR signal corresponded to the PCR product of the expected size. In the case of the absence of a band on the gel but the presence of a SYBR signal, the resulting DNA concentration was set to 0.

mRNA Expression Levels of SEREX-Defined Ovarian Tumor Antigens.

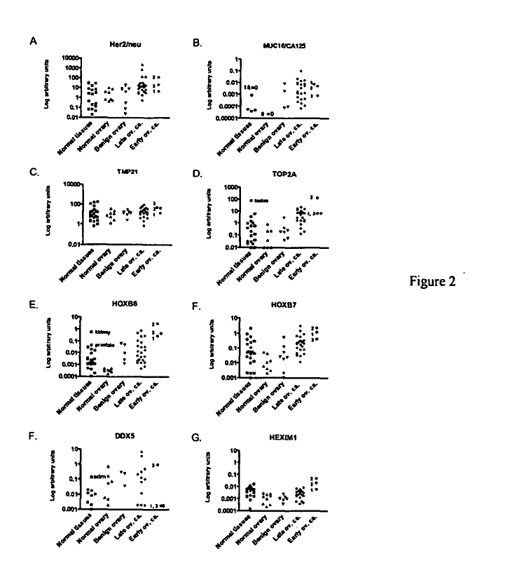

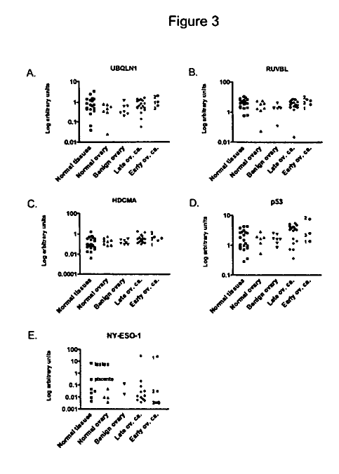

Overexpression of multiple antigens encoded within the HER2/neu cluster, including HER2/neu and TOP2A, has been described in human breast and ovarian tumors. (Tanner et al., Cancer Res. 61:5345-48 (2001); Hengstler et al., Cancer Res. 59:3206-14 (1999); Jarvinen et al., Am. J. Pathol. 156:839-47 (2000).) Therefore, it was investigated whether the genes on chromosome 17q were over-expressed in a similar manner. mRNA expression levels were evaluated across a panel of normal and tumor tissues by semi-quantitative real-time RT-PCR. The tissues used for this analysis were unrelated to the sera used for antibody studies. FIG. 2 compares the relative mRNA expression levels of HER2/neu and the SEREX-derived ovarian tumor antigens encoded on chromosome 17q, including, TOP2A, HOX-B6, Homeobox-B7, DDX5, and HEXIM1. Real-time RT-PCR analysis shows 8% (2/26) of all patients express high levels of mRNA for NY-ESO-1 in tumor tissue (FIG. 3). HER2/neu was also found to be up-regulated at the mRNA level in 19% (5/26) of ovarian tumors. MU16/CA125 is highly expressed in 42% (11/26) of ovarian tumors.

As an additional comparison, the expression of MUC16/CA125, which is reportedly expressed at high levels in ovarian tumors, was analyzed. Expression levels were evaluated in 22 normal tissues (supra), 8 samples of normal whole ovary, 8 samples of benign ovarian tumors, and 26 ovarian cancers (23 late-stage and 3 early stage). The expression level of a housekeeping gene, transmembrane trafficking protein 21 (TMP21), was used as a standard to control for the quality and quantity of cDNA templates (FIG. 2A). Other housekeeping genes such as beta actin (ACTB) and glyceraldehyde-3-phosphate dehydrogenase (GAPDH) showed greater variability in tumors and therefore were not used as controls. The mean and standard deviation of TMP21 values were 29.9/50.4 for normal tissues, 12.8/12.6 for normal ovary, 14.3/18.3 for benign ovary and 22.7/22.7 for ovarian tumors, indicating that the quantity and quality of cDNAs was within about a two-fold range in all groups of tissues analyzed.

Relative to normal ovary, mRNA levels for MUC16/CA125, HER2/neu, TOP2A, HOX-B6, Homeobox-B7 and DDX5 were elevated in multiple ovarian tumors (FIG. 2). These elevations were typically more than one log in magnitude, and thus are unlikely to be attributable to differences in cDNA quantity or quality. Relative to a large panel of normal tissues other than testes, prostate and ovary, only MUC16/CA125, HER2/neu, TOP2A, and DDX5 showed clearly elevated mRNA levels in multiple tumors (FIGS. 2C, D, G). By contrast, HEXIM1 and HOX-B6 were only elevated in one tumor relative to normal tissues. Five other genes from 17q (PYY, PPY, GIP, IGFBP4 and PRAC) that do not encode known tumor antigens also failed to show elevated mRNA levels in ovarian tumors relative to normal tissues.

Notably, the three early stage tumors showed the most consistently elevated mRNA levels for many of these genes. HOX-B6 and HOXB7 are upregulated in ovarian tumors, with some of the highest expression levels observed in early stage tumors (FIG. 2). One early stage tumor showed elevated TOP2A, HER2/neu, HomeoboxB6 and HEXIM1 (FIG. 1, early stage tumor #2). Two other early stage tumors also showed moderately elevated mRNA levels for TOP2A. In fact, the highest expression levels for TOP2A, HOX-B6 and HEXIM1 were observed in early stage tumors. While the mean expression level of TMP21 is somewhat elevated in these early stage tumors, it is within one standard deviation of the mean for normal ovary. The mRNA expression levels for these tumor antigens are beyond this level of variability and thus reflect clear increases in mRNA abundance. These data suggest that HER2/neu, TOP2A, DDX5 and possibly HOX-B6 proteins are overexpressed in a subset of early and late-stage ovarian tumors.

Six of the seven tumors showing the highest levels of HER2/neu mRNA also showed high levels of TOP2A. These data are consistent with several reports showing co-overexpression of HER2/neu and TOP2A in breast and ovarian tumors. (Hengstler et al., Cancer Res. 59:3206-14 (1999); Jarvinen et al., Am. J. Pathol. 156:839-47 (2000).) In addition, five tumors with the highest HOX-B6 mRNA are among the highest expressors of Homeobox-B7. Thus, it appears that genes within this region are coordinately upregulated in some, but not all ovarian tumors.

The other five SEREX-derived antigens, NY-ESO-1, UBQLN1, RUVBL, HDCMA, and p53, are not encoded on chromosome 17q. As with all CT antigens, NY-ESO-1 showed a large increase in mRNA abundance in a subset (2/26) of ovarian tumors, normal testes and placenta compared to all other normal tissues (FIG. 3E). However, in contrast to NY-ESO-1 and the chromosome 17q antigens, the mRNA levels for UBQLN1, RUVBL, HDCMA and p53 were not markedly different in ovarian tumors compared to normal and benign tissues (FIG. 3). Thus, overexpression or mis-expression at the mRNA level is unlikely to be responsible for the induction of autoantibodies to these four antigens. The absence of increased p53 messenger RNA in tumors is consistent with reports correlating mutations in p53 with stabilization of p53 protein and induction of autoantibodies. A similar mechanism may underlie the autoantibody response to UBQLN1, RUVBL and HDCMA.

TOP2A is upregulated in 23% (6/26) of ovarian tumors compared to all normal tissues tested other than testes and prostate. HOX-B6 is upregulated in a subset of tumors, with kidney being the only normal tissue displaying comparable mRNA expression. 27% (7/26) of tumors showed 10 fold elevated HOX-B6 mRNA levels compared to all normal tissues other than kidney. DDX5 showed highly elevated mRNA levels in 12% (3/26) of ovarian tumors compared to normal tissues other than ovary.

The expression patterns of the various tumor antigens described here are largely non-overlapping. If one considers NY-ESO-1, HER2/neu, TOP2A, HOX-B6 and DDX5 together, 50% (13/26) of tumors show elevated mRNA expression for at least one of these antigens. Moreover, if CA125/MUC16 proves to be immunogenic, then 69% (18/26) of tumors would be covered by this panel of target antigens.

Real-time RT-PCR analysis of HER2/neu, TOP2A, HOX-B6, Homeobox-B7, DDX5 and HEXIM1 shows over-expression of mRNA for these antigens in several early stage serous ovarian tumors and not in most normal or benign tissues. Thus, these antigens are be over-expressed and available for immune recognition in a subset of early stage patients.

### Example 5

The tumor associated antigen discovered by SEREX are, by definition, immunogenic in human patients, as the SEREX screening method utilizes patient-derived circulating antibodies to identify tumor antigens. To determine whether patient T cells also recognize a given antigen, lymphocytes are exposed in vitro to the antigen (and appropriate control antigens) and assessed for proliferation, expression of activation markers, cytokine secretion, and/or cytotoxicity against antigen-positive target cells. MHC Class I and II tetramers can also be used to identify T cells that recognize a given antigen or portion thereof (see (See, e.g., U.S. Pat. No. 5,635,363, U.S. patent application Ser. No. 10/116,846; the disclosures of which are incorporated by reference herein.)

The expression pattern a given antigen at the mRNA level can be assessed across a panel of tumors and normal tissues by RNA dot blot, Northern blot, realtime PCR, or in situ hybridization to tissue sections.

Expression patterns at the protein level can be performed using the production of a specific antibody that recognizes the antigen, followed by Western blot or immunohistochemistry with that antibody. The portion of the antigen that is recognized by T cells can be determined for adoptive T cell therapy and tumor vaccination. The recognized epitope can be done by a variety of methods, including computer modeling followed by empirical testing of candidate antigen-derived peptides using the in vitro lymphocyte assays.

Once an tumor associated antigen is identified that shows appropriate immunogenicity and pattern of expression, and that has been formulated into a pharmaceutical-grade reagent that can be used for adoptive T cell therapy or vaccination, an immunotherapeutic composition of T cells or vaccines that target that antigen can be administered to a patient.

### Example 6

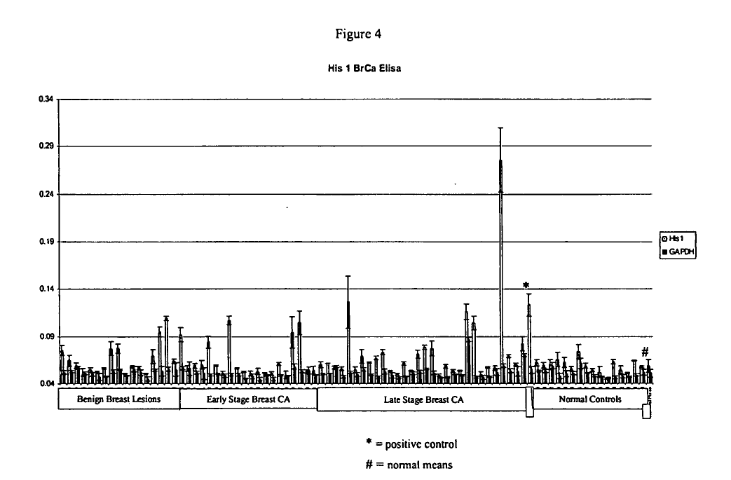

ELISA's of HIS1 and GAPDH protein levels were conducted in samples of benign breast lesions, early stage breast cancer, later stage breast cancer and normal controls. The results are shown in FIG. 4.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

1. A method for screening a subject for ovarian cancer, comprising:
obtaining a blood or serum sample from the subject, the sample comprising antibodies; and
determining whether the sample comprises antibodies to at least one tumor associate antigen selected from Topoisomerase II alpha, Werner helicase interacting protein, HEXIM1, FLJ20267, Deadbox protein-5, and Kinesin-like 6;
wherein the presence of antibodies to the at least one tumor associated antigen is correlated with the presence of ovarian cancer in the subject.

2. The method of claim 1, further comprising:
determining whether the sample comprises antibodies to CA125.

3. The method of claim 1, comprising determining whether the sample comprises antibodies to tumor associated antigens Topoisomerase II alpha, Ubiquilin-1, RUVBL, p53 and NY-ESO-1.

4. The method of claim 3, further comprising:
determining whether the sample comprises antibodies to tumor associated antigen CA125.

5. The method of claim 1, comprising determining whether the sample comprises antibodies to Topoisomerase II alpha, Werner helicase interacting protein, HEXIM1, FLJ20267, Deadbox protein-5, Ubiquilin-1, HOX-B6, p53 and NY-ESO-1.

6. The method of claim 1, comprising determining whether the sample comprises antibodies to at least one of Topoisomerase II alpha, Werner helicase interacting protein, HEXIM1, HDCMA, Deadbox protein-5, and Kinesin-like 6;
optionally at least one of p53, NY-ESO-1 and CA125;
and at least one of ZFP161, Ubiquilin-1, HOX-B6, IFI27, YB-1, KIAA0136, Osteonectin, F-box only protein 21, and ILF3.

7. A method for prognosis or diagnosis of hyperproliferative disease in a subject, comprising:
obtaining a sample from the subject, the sample comprising antibodies;
determining the presence of antibodies in the sample to a tumor associated antigen selected from Topoisomerase II alpha, Werner helicase interacting protein, HEXIM1, FLJ20267, Deadbox protein-5, and Kinesin-like 6, by contacting the sample with the at least one tumor associated antigen; and
detecting complex formation between the tumor associated antigen and the antibodies in the sample;
wherein the presence of antibody to the tumor associated antigen is correlated with the presence of hyperproliferative disease in the subject.

8. The method of claim 7, wherein the subject is a mammal.

9. The method of claim 8, wherein the subject is human.

10. The method of claim 7, wherein the sample is blood, serum, ascites fluid, mucosal fluid, cervical wash, nipple aspirate fluid, stool, urine, saliva, tears, or sputum.

11. The method of claim 10, wherein the sample comprises serum.

12. The method of claim 7, wherein the detecting is by Western blot, radioimmunoassay, ELISA, sandwich immunoassay, immunoprecipitation assay, or protein A immunoassay.

13. The method of claim 7, wherein the hyperproliferative disease is epithelial cancer.

14. The method of claim 13, wherein the epithelial cancer is ovarian cancer, breast cancer, lung cancer or colorectal cancer.

15. A method for prognosis or diagnosis of autoimmune disease in a subject, comprising:
obtaining a sample from the subject, the sample comprising antibodies;
contacting the sample with at least one tumor associated antigen, the tumor associated antigen selected from Topoisomerase II alpha, Werner helicase interacting protein, HEXIM1, FLJ20267, Deadbox protein-5, and Kinesin-like 6; and
detecting complex formation between the tumor associated antigen and the antibodies in the sample;
wherein the presence of antibody to the tumor associated antigen is correlated with the presence of autoimmune disease in the subject.

16. The method of claim 15, wherein the subject is a mammal.

17. The method of claim 16, wherein the subject is human.

18. The method of claim 15, wherein the sample is blood, serum, ascites fluid, mucosal fluid, cervical wash, nipple aspirate fluid, stool, urine, saliva, tears, or sputum.

19. The method of claim 18, wherein the sample comprises serum.

20. The method of claim 15, wherein the detecting is by Western blot, radioimmunoassay, ELISA, sandwich immunoassay, immunoprecipitation assay, or protein A immunoassay.

21. The method of claim 15, wherein the tumor associated antigen further comprises at least one of p53, NY-ESO-1, Ubiquilin-1 and HOX-B6.

22. The method of claim 15, wherein the autoimmune disease is rheumatoid arthritis, graft versus host disease, systemic lupus erythromatosis (SLE), scleroderma, multiple sclerosis, diabetes, organ rejection, inflammatory bowel disease, or psoriasis.

23. A method for prognosis or diagnosis of hyperproliferative disease in a subject, comprising:
obtaining a sample from the subject;
contacting the sample with at least one antibody to a tumor associated antigen selected from Topoisomerase II alpha, Werner helicase interacting protein, HEXIM1, FLJ20267, Deadbox protein-5, and Kinesin-like 6; and
detecting complex formation between the antibody and the tumor associated antigen in the sample;
wherein the presence of to the tumor associated antigen is correlated with the presence of hyperproliferative disease in the subject.

24. The method of claim 23, further comprising:
contacting the sample with antibody to CA125 and detecting complex formation between the antibody and CA125.

25. The method of claim 23, comprising:
contacting the sample with antibodies to Topoisomerase II alpha, Ubiquilin-1, RUVBL, p53 and NY-ESO-1, and detecting complex formation between the antibodies and at least one of Topoisomerase II alpha, Ubiquilin-1, RUVBL, p53 and NY-ESO-1.

26. The method of claim 23, comprising:
contacting the sample with antibodies to Topoisomerase II alpha, Werner helicase interacting protein, HEXIM1, HDCMA, Deadbox protein-5, Ubiquilin-1, HOX-B6, p53 and NY-ESO-1, and detecting complex formation between the antibodies and at least one of Topoisomerase II alpha, Werner helicase interacting protein, HEXIM1, FLJ20267, Deadbox protein-5, Ubiquilin-1, HOX-B6, p53 and NY-ESO-1.

27. The method of claim 23, wherein the subject is a mammal.

28. The method of claim 27, wherein the subject is human.

29. The method of claim 23, wherein the sample is tissue, cells, plasma, serum, ascites fluid, mucosal fluid, cervical wash, nipple aspirate fluid, spinal fluid, lymph fluid, the external sections of the skin, respiratory, intestinal, and genitourinary tracts, tears, saliva, hair, tumors, organs, stool, or urine.

30. The method of claim 23, wherein the detecting is by Western blot, radioimmunoassay, ELISA, sandwich immunoassay, immunoprecipitation assay, or protein A immunoassay.

31. The method of claim 23, wherein the hyperproliferative disease is epithelial cancer.

32. The method of claim 31, wherein the epithelial cancer is ovarian cancer, breast cancer, lung cancer or colorectal cancer.

33. A method for prognosis or diagnosis of hyperproliferative disease in a subject, comprising:
contacting an array of probe molecules stably associated with a surface of a solid support with a sample comprising target nucleic acids under hybridization conditions sufficient to produce a hybridization pattern,
the probe molecules comprising nucleic acids encoding at least a fragment of at least one of Topoisomerase II alpha, Werner helicase interacting protein, HEXIM1, FLJ20267, Deadbox protein-5, and Kinesin-like 6;
detecting the hybridization pattern; and
determining whether the subject has the hyperproliferative disease.

34. The method of claim 33, wherein the probe molecules comprise nucleic acids encoding at least a fragment of least one of Topoisomerase II alpha, Werner helicase interacting protein, HEXIM1, HDCMA, Deadbox protein-5, and Kinesin-like 6;
optionally at least one of p53, NY-ESO-1 and CA125;
and at least one of ZFP161, Ubiquilin-1, HOX-B6, IFI27, YB-1, KIAA0136, Osteonectin, F-box only protein 21, and ILF3.

35. The method of claim 33, wherein the sample is from ovary, lung, breast or a colorectal tract of the subject.

36. The method of claim 33, wherein the sample is tissue, cells, plasma, serum, ascites fluid, mucosal fluid, cervical wash, nipple aspirate fluid, spinal fluid, lymph fluid, the external sections of the skin, respiratory, intestinal, and genitourinary tracts, tears, saliva, hair, tumors, organs, stool, or urine.

37. The method of claim 33, wherein the hyperproliferative disease is epithelial cancer.

38. The method of claim 37, wherein the epithelial cancer is ovarian cancer, lung cancer, breast cancer or colorectal cancer.

39. The method of claim 33, wherein the target nucleic acids are labeled.

40. A method for prognosis or diagnosis of hyperproliferative disease in a subject, comprising:
obtaining a nucleic acid-containing sample from the subject;
determining a methylation profile for a tumor associated antigen gene in the sample, wherein the tumor associated antigen gene encodes Topoisomerase II alpha, Werner helicase interacting protein, HEXIM1, FLJ20267, Deadbox protein-5, or Kinesin-like 6;
comparing the methylation profile of the tumor associated antigen gene with a known methylation profile for the tumor associated antigen gene; and
prognosing or diagnosing a risk of hyperproliferative disease in the subject.

41. The method of claim 40, wherein the methylation profile is determined by contacting the nucleic acid-containing sample with an agent that modifies unmethylated cytosine,
amplifying the nucleic acid in the sample with oligonucleotide primers that hybridize with the tumor associated antigen gene sequence, the oligonucleotide primers distinguishing between modified methylated and non-methylated nucleic acid; and
detecting the methylated nucleic acid based on the presence or absence of amplification products produced in the amplification step.

42. The method of claim 41, wherein the amplifying step is by polymerase chain reaction.

43. The method of claim 41, wherein the modifying agent is bisulfite.

44. The method of claim 41, wherein the oligonucleotide primers amplify the promoter region of the tumor associated antigen gene.

45. The method of claim 40, wherein determination of the methylation profile comprises methylation-sensitive restriction enzyme digestion.

46. The method of claim 40, wherein determination of the methylation profile comprises methylation-dependent restriction enzyme digestion.

47. The method of claim 40, further comprising:
determining the methylation profile of a gene encoding p53, NY-ESO-1 or CA125.

48. The method of claim 40, further comprising:
determining the methylation profile of a second gene encoding ZFP161, Ubiquilin-1, HOX-B6, IFI27, YB-1, KIAA0136, Osteonectin, F-box only protein 21, or ILF3; and
optionally determining the methylation profile of a third gene encoding p53, NY-ESO-1 or CA125.

49. A kit for detecting antibodies to a tumor associated antigen, comprising:
at least one tumor associated antigen selected from Topoisomerase II alpha, Werner helicase interacting protein, HEXIM1, FLJ20267, Deadbox protein-5, and Kinesin-like 6; and
anti-human antibody.

50. The kit of claim 49, wherein the tumor associated antigen is labeled.

51. The kit of claim 49, wherein the anti-human antibody is labeled.

52. The kit of claim 49, further comprising at least one of ZFP161, Ubiquilin-1, HOX-B6, IFI27, YB-1, KIAA0136, Osteonectin, F-box only protein 21, and ILF3, and optionally at least one of p53, NY-ESO-1 and CA125.

53. A kit for detecting antibodies to a tumor associated antigen, comprising:
Topoisomerase II alpha, RUVBL, p53 and NY-ESO-1.

54. The kit of claim 53, further comprising:
at least one of Ubiquilin-1 and CA125.

55. A kit for detecting expression of tumor associated antigen genes, comprising:
nucleic acid primers to a tumor associated antigen nucleic acid, the tumor associated antigen nucleic acid selected from at least one of Topoisomerase II alpha, Werner helicase interacting protein, HEXIM1, FLJ20267, Deadbox protein-5, and Kinesin-like 6;
and a polynucleotide polymerase.

56. The kit of claim 55, wherein said kit further comprises nucleotides.

57. The kit of claim 55, further comprising a buffer.

58. The kit of claim 55, further comprising an array of probe molecules for use in a hybridization assay.

59. A method for treating a subject having a hyperproliferative disease, comprising:
administering to the subject an effective amount of an immunotherapeutic composition effective to reduce or ameliorate the hyperproliferative disease,
the immunotherapeutic composition comprising:
at least one of Topoisomerase II alpha, Werner helicase interacting protein, HEXIM1, FLJ20267, Deadbox protein-5 and Kinesin-like 6;
antibodies to at least one of Topoisomerase II alpha, Werner helicase interacting protein, HEXIM1, FLJ20267, Deadbox protein-5 and Kinesin-like 6;
antigen presenting cells loaded with at least one of Topoisomerase II alpha, Werner helicase interacting protein, HEXIM1, FLJ20267, Deadbox protein-5 and Kinesin-like 6, or a fragment thereof; or
T cells loaded with at least one of Topoisomerase II alpha, Werner helicase interacting protein, HEXIM1, FLJ20267, Deadbox protein-5 and Kinesin-like 6, or a fragment thereof.


SEQUENCE LISTING (28 sequences)

SEQ ID NO: 1 - DNA, 18 bp, Artificial Sequence
  Sequencing Primer
tccgagatctggacgagc

SEQ ID NO: 2 - DNA, 20 bp, Artificial Sequence
  Sequencing Primer
taatacgactcactataggg

SEQ ID NO: 3 - DNA, 24 bp, homo sapiens
atgactaccctaactcctcagagg

SEQ ID NO: 4 - DNA, 24 bp, homo sapiens
aagcaggctagagtaacctctgtc

SEQ ID NO: 5 - DNA, 24 bp, homo sapiens
ccaacagggaagagtgtcgcaccc

SEQ ID NO: 6 - DNA, 24 bp, homo sapiens
actgcttgagcatcctcaggagct

SEQ ID NO: 7 - DNA, 24 bp, homo sapiens
ctctgagactgatggctacgttgc

SEQ ID NO: 8 - DNA, 24 bp, homo sapiens
cgtctttgacgaccccattcttcc

SEQ ID NO: 9 - DNA, 24 bp, homo sapiens
cgatgacgacttcatggaagaagg

SEQ ID NO: 10 - DNA, 27 bp, homo sapiens
actccttgatgagctcctgcttggctc

SEQ ID NO: 11 - DNA, 24 bp, homo sapiens
cactccggtctacccgtggatgca

SEQ ID NO: 12 - DNA, 26 bp, homo sapiens
catatcttgatctgcctctccgtcag

SEQ ID NO: 13 - DNA, 24 bp, homo sapiens
cactccggtctacccgtggatgca

SEQ ID NO: 14 - DNA, 24 bp, homo sapiens
atctgtctttccgtgaggcagagc

SEQ ID NO: 15 - DNA, 21 bp, homo sapiens
gccactggctcggagagtaga

SEQ ID NO: 16 - DNA, 20 bp, homo sapiens
ccggcaagttccagtcattg

SEQ ID NO: 17 - DNA, 25 bp, homo sapiens
ggtgcttctgaaggagttcactgtg

SEQ ID NO: 18 - DNA, 24 bp, homo sapiens
ttagcgcctctgccctgagggagg

SEQ ID NO: 19 - DNA, 24 bp, homo sapiens
cactgcccaacaacaccagctcct

SEQ ID NO: 20 - DNA, 25 bp, homo sapiens
gtctgagtcaggcccttctgtcttg

SEQ ID NO: 21 - DNA, 24 bp, homo sapiens
ctgcgttaacacaagcggttgctg

SEQ ID NO: 22 - DNA, 24 bp, homo sapiens
cccaaatccttcatcagcctagtg

SEQ ID NO: 23 - DNA, 20 bp, homo sapiens
cgacgcttcttcaaggccaa

SEQ ID NO: 24 - DNA, 20 bp, homo sapiens
atggaagcccaagctgctga

SEQ ID NO: 25 - DNA, 24 bp, homo sapiens
ctgagtcttccacctccactaccg

SEQ ID NO: 26 - DNA, 24 bp, homo sapiens
tgcccgaggagccacagctgagtc

SEQ ID NO: 27 - DNA, 24 bp, homo sapiens
cagttattcagcagatgctgcagg

SEQ ID NO: 28 - DNA, 26 bp, homo sapiens
gagcccagtaacctttcaatagctgc